(19) United States
(12) Patent Application Publication
(10) Pub. No.: US 2024/0018491 A1
(43) Pub. Date: Jan. 18, 2024

(54) Purified Immobilized Lipases

(71) Applicant: Enzymocore Ltd., Shfaram (IL)

(72) Inventors: Sobhi Basheer, Sakhnine (IL); Ramez Masri, Nahef (IL)

(21) Appl. No.: 18/039,520

(22) PCT Filed: Dec. 1, 2021
(86) PCT No.: PCT/IL2021/051432

§ 371 Date: May 31, 2023

Related U.S. Application Data
(60) Provisional application No. 63/119,986, filed on Dec. 1, 2020.

(51) Int. Cl. C12N 9/20; C12P 7/6458; C12N 11/08

(52) U.S. Cl. CPC C12N 9/20; C12Y 301/01003; C12P 7/6458; C12N 11/08

(57) ABSTRACT

Disclosed are methods for purifying immobilized lipase preparations from various pigments and colorants as well as other contaminants, using various lipase-catalyzed reactions with oils and fats as substrates, such as esterification, transesterification and interesterification reactions. Also disclosed are purified immobilized lipase preparations and their industrial uses.

### TECHNOLOGICAL FIELD

The present disclosure generally relates to purified immobilized lipase preparations, methods for their production and uses thereof.

### BACKGROUND ART

References considered to be relevant as background to the presently disclosed subject matter are listed below:
[1] Melani, N. et al., (2019) Lipases: From Production to Applications, Separation & Purification Reviews, P. 1-19
[2] Guerrand, D., Lipases industrial applications: Focus on food and agroindustries. 2017. OCL Oilseeds and fats crops and lipids, EDP, 2017, 24 (4), pp. D403
[3] V. Gotor, et al., 1991. Tetrahedron, Vol. 47(44), p. 9207-9214)
[4] U.S. Pat. No. 6,528,293 B1
[5] Ventura, S. P. M., & Coutinho, J. A. P. (2016), 59-97, AOCS PRESS
[6] Junker B. Biotechnol. Prog. 2007, 23, P. 767-784
[7] U.S. Pat. No. 6,605,452 B1
[8] U.S. Pat. No. 8,551,743 B2
[9] U.S. Pat. No. 8,580,550 B2
[10] WO 2008/027527 A2
[11] U.S. Pat. No. 8,357,814 B2
[12] U.S. Pat. No. 9,896,703 B2
[13] US 2007/0264695 A1
   Acknowledgement of the above references herein is not to be inferred as meaning that these are in any way relevant to the patentability of the presently disclosed subject matter.

### BACKGROUND

Oils and fats that comprise various organic compounds, in general gain color when treated with crude or immobilized lipases or phospholipases, so as to catalyze different reactions such as esterification, transesterification, interesterification, hydrolysis, amidation and transamidation, as well as others. Pigments and many other fermentation-aid ingredients, such as organo-silicon compounds, are typically concentrated together with crude lipases. When the crude lipases are adsorbed on polymeric supports during enzyme immobilization processes, the pigments and other fermentation-aid agents are also adsorbed. The presence of pigments and other fermentation agents in the crude or immobilized lipases entails coloration implications of the final product of the enzymatic process, desorption of impurities to the reaction media as well as decay of the enzyme activity. The use of many enzymes in their crude as well as immobilized forms, in particularly lipases, is restricted in many industrial scale processes.

### Introduction

Lipases (triacylglycerol hydrolases, E.C. 3.1.1.3) in their natural form hydrolyze the ester linkage of triglyceride molecules at the water-oil interface to yield free fatty acids, partial glycerides and glycerol, where the ratio between these products depends on the source, regio-specificity and substrate selectivity of the enzyme. The substrates of lipases are mainly neutral lipids, to some extent polar lipids especially when phospholipases are involved, such as phospho-, ether- and glyco-lipids, and possibly organic molecules which contain functional groups including ester, carboxylic, hydroxyl, amine and amide groups. During the last two decades lipases and phospholipases have been widely used at industrial scales in many fields including [1]:
1. Fats and oils processing through interesterification (for the production of modified acylglycerols as an alternative for alkali metal alkoxides-catalyzed interesterification for the production of homogeneous oils and fats blends); acidolysis (for the production of cocoa butter equivalent and human milkfat substitute); transesterification (for the production of fatty acids short-chain alkyl esters defined as biodiesel); esterification (for the production of wax esters and other inedible and food-grade esters); and hydrolysis (for the production of free fatty acids and partial glycerides, and/or glycerol, as well as hydrolysis of phospholipids by phospholipases in oil degumming processes) [2].
2. Fatty acids (omega-3 and omega-6) concentrates from oils of different sources, including fish and oleaginous microorganisms through the employment of the different selectivity of lipases toward their different substrates.
3. Production of pharmaceuticals and cosmeceuticals through catalyzing different reactions including esterification, transesterification, interesterification, amidation and transamidation, in particularly for the production of chemically instable organic compounds, water-intolerant organic compounds and optically active intermediates [3].
4. Production of flavors and fragrances through different catalyzed reactions.
5. Pretreatment of lipid-rich wastewater for hydrolyzing lipids prior to their discharge into municipal wastewater treatment units.
6. Leather, paper, detergents and textile industries for removal of fats and oils.

Preparation of industrial enzymes in general and lipases in particular, involves separation of cells or cell debris (in case the enzyme is intracellular) from the fermentation broth as a first stage of the process. Such process is normally carried out by common techniques including the use of centrifugation and filtration. In order to obtain lipase concentrates in liquid or solid preparations different techniques might be applied on the fermentation broth after removal of cells and/or cell debris. Such techniques include the following subsequent purification steps:
1. Use of ammonium sulfate for the precipitation and removal of contaminant proteins.
2. Dialysis for the removal of low size contaminants, e.g., salts.
3. Applying high resolution purification technique such as crystallization, ultra- and nano-filtration membranes, ion- and gel- chromatography, fractional precipitation by solvents e.g., acetone or ethanol, and fractional liquid-liquid extraction.
4. Lipase concentrates after application of the former stages may be used “as is” or diluted with solid (such as cyclodextrins and lactose) or liquid (glycerol, and glycols in water solutions) excipients.
5. Further purified and activated lipases may be prepared by placing crude enzymes in fat-and-water boundary surfaces urging lipases to adopt their active conformation at the interface of the oil and water phases. Purified and activated lipases at the interface can be collected after the removal of both phases followed by application of freeze-drying [4].

Many commercial lipase preparations have been prepared following one or two stages of the above recommended procedures aiming at reducing production costs and make their commercial application economically affordable. For example, it has been reported [5] that lipase from Burkholderia pseudomallei can be purified by a factor of 13.4 and a yield of 99% using a single-step purification technique when applying solvents, such as ethanol and isopropanol in combination with salts in the purification process. Regardless of the type of the techniques applied in enzyme purification processes, in many cases lipases might drag different pigment ingredients and other contaminants such as organic silicon compounds normally used as antifoam agents in fermentation processes [6] through the whole applied above-mentioned common techniques and end up in the final industrial lipase preparations.

Preparation of industrial enzymes in general, and lipases in particular, involves separation of cells or cell debris (in case the enzyme is intracellular) from the fermentation broth.

The market demand for lipases is huge and very much diversified, however, because of their high-cost contribution in the production process their consumption in different industries remains economically prohibited in many processes Immobilization of enzymes on recyclable beads is considered as potential tool, in order to reduce enzyme cost contribution in the overall process Immobilization methods of lipases include adsorption on polymeric organic and in inorganic supports, adsorption on ion-exchange resins, covalent binding on activated surfaces of organic and inorganic supports and cross-linking of lipases to form recyclable crystals or aggregates. The utilization of immobilized enzymes facilitates the use of conventional industrial scale reactors, such as stirred tank reactors operated batch-wise or continuously, fixed-bed, and fluidized-bed reactors. Different materials might be added during the immobilization process to be part of the final immobilized enzyme preparation, so as to increase the activity as well as operational stability of lipases in various industrial applications. Such additives include sugar fatty acids esters ([7], [8]), fatty acids, polyethylene glycol, medium-chain fatty acids, an alcohol ester (e.g., monoglycerides) or a mixture of both components [9], and calcium and magnesium oxides. The final immobilized enzyme preparation can be in the form of beads or granulated in the form of powder.

When lipases are immobilized on inorganic or organic supports, pigments and other contaminants such as organic silicon compounds, originally come from the fermentation broth would also adhere on the surface of the enzyme-hosting support. Depending on the physical and chemical nature of such adsorbed pigments and other contaminants, when using immobilized lipases for production purposes such coloring agents and contaminants, in addition to migration of specific resin constituents, such as monomers or oligomers of the polymeric resin support might be released into the reaction medium and result in coloring as well as contaminating of the final product [4].

Interesterification of oils and fats involves interchanging of esters in and/or among the triglyceride molecules comprising oil and fats which results in producing modified fats and oil of desired physic-chemical characteristics. The exchange of fatty acyl groups between triglycerides molecules comprising any oil and fat is typically catalyzed by a strong alkaline catalyst, such as sodium/potassium methoxide, or a lipase in its free or immobilized forms. Unlike conventional alkali chemical catalysts, depending on the source of the enzyme, lipases may catalyze the exchange of fatty acyl groups on the glycerol backbone of different triglycerides randomly, or using lipase/s with sn-1,3-positional specificity, creating a major advantage for the production of restructured triglycerides for specific applications, including production of human milk fat substitutes and cocoa butter equivalents. Regardless of the type of catalyst, interesterification has been practiced widely during the last three decades as an alternative for the partial hydrogenation process for the production of functional fats and oils. Soaps produced as a byproduct of the chemical/enzymatic interesterification process as well as the residual catalyst and coloring agents produced through the process or desorbed from the catalyst in the treated oils and fats must be removed after the interesterification process. Chemically interesterified oils and fats are normally purified by the addition of water, typically, 0.3-5% by weight of oil, in order to inactivate the alkali metal alkoxides used catalyst, followed by centrifugation for removal of the water phase containing the inactivated catalyst and part of the soaps produced as a byproduct of the process. When using enzymes as catalysts in the interesterification process, normally oils and fats are pretreated with an adsorbent which has been approved to be as an essential step for the removal of hydroperoxides, metal ions and secondary oxidation products (e.g., aldehydes and ketones) in order to expand the operational lifetime of the

biocatalyst in the process [10].

The resulting reformed oils and fats, after chemical/enzymatic interesterification, are normally further treated with an adsorbent, such as an activated clay, activated carbon or silica-magnesia, in order to remove the residual soaps and alkali metal ions as well as remove other coloring agents co-generated in the interesterification process. This process is normally carried out by adding an adsorbent, typically 0.1-5% wt. to the oil and mixing at 70-110° C. for 15-60 minutes, and then filtration of the oil. Prior to or after treatment of interesterified oils and fats with an adsorbent, washing of the oil with citric acid solution might also be conducted in order to remove heavy metals as well as convert the residual soaps to free fatty acids [11]. Decolorized oil/fat is then subjected to deodorization process with steam at 200-240° C. under reduced pressure to obtain the purified oil. It has been shown that the activity of lipases cannot be stabilized by decolorization step alone, in which fat and/or oil and clay are brought in contact with each other at a high temperature of 90-150° C. However, it has been demonstrated that when a mixture of oils and/or fats is pretreated with a clay at low temperature and under conditions different from those for decolorization, typically 30-80° C., before treatment with a lipase in order to catalyze the interesterification process, the decay in the activity of the enzyme has been greatly lowered [12]. Though these oil purification methods are practiced worldwide for processing oils and fats, there still are persistent pigment components and other impurities that desorb from used enzyme preparations to the oil and cannot be removed from the final product.

In order to avoid coloration and accumulation of other contaminants in lipase-treated fats and oils, commercial lipases have been purified by a two stage-process comprising of: (a) bringing a long chain fatty acid triglycerides and medium-chain triglycerides into contact with a lipase, and (b) collecting the purified lipase by filtration. According to this production method, the purified lipase can be obtained whereas content of impurities such as organo-silicon compounds and pigments ingredients, as well as specific constituents of the support polymeric resin/s, such as monomers or oligomers, migrated from the resin into the reaction system, are decreased. Such purified lipases can be used for producing a fat and fatty oil composition which comprises a step of exchanging esters of the fat and fatty oil. Such lipases can be used in powdery form which is not immobilized to a carrier or immobilized to a carrier such as anion-exchange resins, phenol absorbing resins, hydrophobic carriers, cation exchange resins, and chelate resins. In the enzyme purification process, in addition to triglycerides oil a partial ester of fatty acid of glycerin and/or a partial ester of a fatty acid of glycerin condensation may be brought into contact with a lipase [13]. It has been demonstrated that such application is insufficient for producing immobilized lipases that do not desorb pigment ingredients and other impurities into the treated oils and fats products. Wasting of large volumes of enzymatically processed food-grade oils and fats with high color extent/intensity occurs very often due to desorption of pigment components and other specific polymer resin support constituents, such as monomers or oligomer, from the used biocatalyst to the final products, which leads to major economic losses.

### GENERAL DESCRIPTION

Disclosed herein is a method for purifying an immobilized lipase preparation, the method comprising a reaction cycle that comprises the steps of (1) reacting a reaction substrate comprising at least one fatty acid source comprising oils, glycerides, free fatty acids and/or fatty acid alkyl esters (FAAE) with an alkyl alcohol, in the presence of at least 100 ppm water or aqueous alkaline buffer solution and a lipase preparation in an immobilized form, to yield fatty acid alkyl esters, partial glycerides and glycerol, allowing the reaction to proceed until at least a part of the reaction substrate, for example, but not limited to at least 10%, 20%, 30%, 40% or 50% of the reaction substrate is converted to FAAE, (2) collecting the medium by filtration to obtain a purified immobilized lipase preparation, and optionally washing the purified immobilized lipase preparation with a suitable solvent, and optionally repeating the reaction cycle comprising said steps (1) and (2) for from 1-10 additional times (1-10 subsequent cycles), using a fresh reaction substrate for each cycle, while using the same batch of immobilized enzyme preparation in all said cycles.

Also disclosed herein is method for purifying an immobilized lipase preparation comprising at least one reaction cycle, the reaction cycle comprising the steps of (a) providing a reaction substrate comprising at least one fatty acid source, specifically at least one of oils, fats, glycerides, free fatty acids and fatty acid alkyl esters; (b) adding to said reaction substrate water or aqueous alkaline buffer at more than 100 ppm up to 70% w/w to form a reaction medium; (c) providing an immobilized lipase preparation comprising a lipase immobilized on an organic or inorganic support; (d) adding the said lipase preparation to said reaction medium to form a reaction mixture, wherein the pH of the reaction medium is a pH of 4-11 and wherein the temperature of the reaction mixture is 10-50° C.; (e) subjecting said fatty acid source to alcoholysis by stepwise adding to said reaction mixture an alkyl alcohol, at a molar ratio of at least 2:1 between said alcohol and said reaction substrate, and stiffing or shaking or recirculating the resulting mixture until at least a part of said reaction substrate, such as at least 10%, 20%, 30%, 40% or 50% of said reaction substrate are converted to fatty acid alkyl esters; (f) adding alkyl alcohol to the reaction mixture of step (e) to reach at least a total molar ratio of 3:1 for hydrophobic or mild hydrophobic polymer resin support, and less than 2:1 for hydrophilic polymer resins used as carrier/support for the immobilization of the enzyme, between said alcohol and said reaction substrate, and allowing the reaction to proceed until conversion of said reaction substrate to fatty acid alkyl esters exceeds 70%, specifically for hydrophobic polymer resin, and less than 70%, specifically for hydrophilic polymer resins used as carrier for the immobilization of the enzyme; (g) collecting the reaction medium of step (f) by filtration and keeping the immobilized lipase preparation, and optionally washing the immobilized lipase preparation with a suitable solvent; wherein the collected reaction medium filtrate separates into two phases, an upper organic/oil phase comprising the formed fatty acid alkyl esters preferably at more than 50% w/w, free fatty acids and mono-, di- and residual tri-glycerides preferably at less than 10% w/w, and a lower (heavier) phase comprising the formed glycerol and water, wherein the upper oil/organic phase comprises extracted hydrophobic pigment components and other hydrophobic contaminants comprised in the said immobilized lipase preparation, such silicon compounds and/or desorbed components of the support, the lower phase comprises extracted hydrophilic pigment components and other hydrophilic contaminants comprised in the said immobilized lipase preparation, and any reaction intermediate products including free fatty acids, fatty acids soaps, mono- and di-glycerides comprise extracted amphiphilic pigment components and other amphiphilic contaminant comprised in the said immobilized lipase preparation; and measuring the color intensity in said upper oil phase and in said lower phase by suitable means; (h) repeating said reaction cycle comprising said steps (a)-(g) at least once as a subsequent (or second) identical reaction cycle, preferably repeating 1-10 times, using in each cycle the same batch of said lipase preparation provided in step (c) of said first reaction cycle, with fresh reaction substrate and alkyl alcohol in each cycle, until the color intensity (expressed, for example, as optical density (OD)) of the oil phase of said filtrate is reduced to a desired level as compared to the color intensity of the oil phase obtained in said first reaction cycle, wherein the immobilized lipase collected after the at least one subsequent reaction cycle is of higher purity and of at least comparable or improved enzymatic activity to that of the immobilized lipase preparation provided in step (c) of said first cycle.

In the above methods, the fatty acid source can be, but is not limited to, at least one oil, such as a plant oil such as soybean oil, canola oil, rapeseed oil, olive oil, MCT oil, castor oil, palm oil, sunflower oil, safflower oil, peanut oil, cotton seed oil, Jatropha oil, coconut oil or corn oil; algal oil, fish oil, oleaginous microorganisms derived oil; waste cooking oil; and any mixtures thereof; said fat is animal-derived fat or brown grease; said free fatty acids are saturated or unsaturated fatty acids of 12-20 carbon atoms, such as mono- or polyunsaturated fatty acids and short and medium-chain fatty acids of 2-12 carbon atoms; said glycerides are mono-, di- and triglycerides of short-, medium- and long-chain fatty acids of 12-20 carbon atoms and their mixtures at any ratio; and said fatty acid alkyl esters are at least one of methyl, ethyl or longer alkyl fatty acid esters, wax esters and sterol esters.

In the above methods the said alkyl alcohol can be, but is not limited to, short-chain C₁₋₆ alkyl alcohol, preferably ethanol, medium-chain C₈₋₁₂ alkyl alcohol, or long-chain C₁₄₋₂₂ alkyl alcohol.

In the above methods, the said lipase can be, but is not limited to, any of a lipase derived from Rhizomucor miehei, Pseudomonas sp., Pseudomonas cepacia, Pseudomonas fluorescens, Rhizopus niveus, Mucor javanicus, Rhizopus oryzae, Aspergillus niger, Penicillium camembertii, Burkholderia ubonensis (strain PL266-QLM also referred to herein as “Lipase QLM”) and cepacia, Alcaligenes sp., Acromobacter sp., Burkholderia sp., Thermomyces lanuginosa, Humicola lanuginosus, Chromobacterium viscosum, Candida antarctica B, Hyphozyma sp., Candida parapsilosis, Candida rugosa, Candida antarctica A, H. insolens, Crytococcus spp., Geotricum candidum Pseudomonas (Burkholderia) cepacia, Pseudomonas stutzeri or papaya seeds, and pancreatin.

In the above methods, said lipase can be, but is not limited to, a random or sn-1,3 positional specific lipase. Further, the said lipase can possess selectivity toward a specific type of fatty acids such as short-, medium- and long-chain fatty acids, or saturated, mono-unsaturated or polyunsaturated fatty acids.

In the above methods, the said support can be, but is not limited to, an anion- or cation-exchange resin, a hydrophilic organic polymer, such as polyacrylate, polymethyl methacrylate, polymethyl methacrylate or cross-linked phenol formaldehyde condensate, or hydrophobic/mild hydrophobic organic polymer such as polyvinyl alcohol, polydivinyl-benzene and polystyrene, and any mixture thereof, an inorganic support such as silica, Celite, diatomaceous earth and perlite. More specifically, the said support can be an anion-or cation-exchange resin, a hydrophilic organic polymer, such as polyacrylate, polymethyl methacrylate, polymethyl methacrylate or cross-linked phenol formaldehyde condensate, or hydrophobic organic polymer such as polyvinyl alcohol, polydivinylbenzene and polystyrene, and any mixture thereof and said desorbed component of the support is a monomer or oligomers of said resin/polymer.

The said immobilized lipase preparation can be, but is not limited to, in powder form with particle size of 1-100 microns, or in the form of beads, typically of 0.01-2 mm diameter. The immobilized lipase is added to said reaction medium at 0.1-20% wt. based on weight of said fatty acid source, such as 1, 2, 3, 4, 5, 6, 7, 10 and up to 20% wt.

The said aqueous alkaline buffer can be, but is not limited to, a bicarbonate, carbonate, acetate, phosphate, citrate or tris, buffer salt or any of their combination. In the above methods, the said reaction substrates can contain said water or aqueous alkaline buffer at an amount of from about 100 ppm to about 70% by weight but is not limited thereto. Exemplary amounts of water or aqueous alkaline buffer added to the reaction substrate are amounts of from about 1%, 2%, 3%, 4%, 5%, 10%, or 20% of weight of said fatty acid source.

In the above methods, the fat source can comprise oils and the products of the enzymatic transesterification reaction are fatty acid alkyl esters and glycerol as byproduct.

In the above methods, the fatty acid source can comprise free fatty acids, and the products of the enzymatic esterification reaction are fatty acid alkyl esters and water as byproduct.

In the above methods, the fatty acid source can comprise fatty acids alkyl esters, and the alcoholysis reaction products are different fatty acids alkyl esters and an alcohol as a byproduct.

In the above methods, the fatty acid source can comprise a mixture of fatty acids and triglycerides, at any ratio, and the esterification/transesterification reaction products are fatty acids alkyl esters, and glycerol and water as byproducts.

In the above methods, the fatty acid source can be a mixture of fatty acids and mono-, di-, and tri-glycerides at any ratio, and the esterification/transesterification reaction products are fatty acids alkyl esters, and glycerol and water as byproducts.

In the above methods, the fatty acid source can be an isolated mono-, di-, or tri-glyceride, or any mixture of at least two thereof, and the transesterification reaction products are fatty acids alkyl esters, and glycerol as byproduct.

In the above methods, the fatty acid source can comprise a mixture of triglycerides, free fatty acids and lecithin gum, at any ratio, and the esterification/transesterification reaction products are fatty acids alkyl esters, and glycerol water, lyso-phospholipids and glycerophospholipids as byproducts.

In the above methods, the said color intensity of the said upper oil phase, if required after dilution with an organic solvent, can be expressed by optical density at 420 nm, but is not limited thereto. The dilution of oil phase: solvent can be, for example, at a ratio of 0-10 v/v, depending on the optical density. The diluent organic solvent can be, but is not limited to any one of n-hexane, iso-propanol, n-propanol, n-butanol, iso-butanol and tert-butanol.

In the above methods, the said steps (a)-(g) can be repeated from 2 to 3 times, 2 to 4 times, 2 to 5 times, 2 to 6 times and up to about 10 times or more.

Further disclosed herein are purified immobilized lipase preparations obtained by any of the above-described as well as other methods presented herein.

Further disclosed herein are purified immobilized lipase preparations, in which the lipase is immobilized on a macroporous resin polymer that is a hydrophobic polymer, a mild hydrophobic polymer or a mixed hydrophobic/hydrophilic polymer, wherein the optical density of the product of a reaction between a fatty acid source and an alcohol in a reaction medium containing water or alkaline buffer in the presence of said purified lipase is reduced compared to optical density of the product of the same reaction carried out in the presence of an identical immobilized lipase preparation that is not purified.

A specific non-limiting example of a purified immobilized lipase as disclosed herein is a Lipase QLM preparation in which the Lipase QLM is immobilized on a macroporous resin polymer that is a hydrophobic polymer, a mild hydrophobic polymer or a mixed hydrophobic/hydrophilic polymer, and the optical absorbance at 420 nm of the oil phase of the product of soybean oil treated by one reaction 24 hours cycle with ethanol at an oil to ethanol molar ratio of 1:3 in the presence of said immobilized lipase at a concentration of 10% w/w is 0.150 OD, and 0.100 OD after three reaction cycles each of 24 hours using the same batch of said immobilized lipase, compared to optical density of 0.750 OD and 0.360 OD for soybean oil mixed with 10% w/w immobilized Lipase QLM preparation for a first cycle of 24 hours and for three cycles of 24 hours each with same batch of lipase. For this specific purified immobilized Lipase QLM preparation, the optical density (OD) at 420 nm of the oil phase of the product of MCT oil treated by one reaction cycle of 24 hours with ethanol at an oil to ethanol molar ratio of 1:3, in the presence of said immobilized lipase at a concentration of 10% w/w is 0.150 OD and 0.050 OD after three reaction cycles each 24 hours when using the same batch of immobilized enzyme, compared to 0.530 OD and 0.082 OD for MCT oil mixed with 10% w/w immobilized Lipase QLM for a first cycle of 24 hours and for three cycles each of 24 hours and each with the same batch of lipase, and the optical density at 420 nm of the oil phase of the product of fish oil treated by one reaction cycle of 24 hours with ethanol at an oil to ethanol molar ratio of 1:3 in the presence of said immobilized lipase at a concentration of 10% w/w is 0.70 OD and 0.32 OD after three reaction cycles each 24 hours when using the same batch of immobilized enzyme, compared to 0.86 OD and 0.50 OD for fish oil mixed with 10% w/w immobilized lipase for a first 24 hours cycles and for three 24 hours cycles each with the same batch of lipase.

Further disclosed herein is a purified immobilized Lipase QLM preparation in which the lipase is immobilized on a macroporous resin polymer composed on polymethyl methacrylate crosslinked with divinyl benzene (DVB) having the FTIR spectra designated (2), (3), (4) or (5) in FIG. 11 and/or in FIG. 12.

In specific non-limiting examples of purified immobilized lipase preparations of the present disclosure the said lipase can be any one of Rhizomucor miehei, Pseudomonas sp., Pseudomonas cepacia, Pseudomonas fluorescens, Burkholderia ubonensis (strain PL266-QLM, also referred to herein as Lipase QLM) and cepacia, Alcaligenes sp., Burkholderia sp., Thermomyces lanuginosa, Humicola lanuginosus, Candida antarctica B, Hyphozyma sp., Candida parapsilosis, Candida antarctica A, Pseudomonas (Burkholderia) cepacia and Pseudomonas stutzeri, immobilized on a hydrophobic linear or branched aromatic or hydrophobic aliphatic polymer-based support.

The lipase in the disclosed purified preparations can be immobilized on a mild hydrophobic polymer, for example divinylbenzene, or on a mixed hydrophobic/hydrophilic polymer, for example cross linked divinylbenzene-methyl methacrylate polymer.

Purified immobilized lipase preparations according to the present disclosure can be used, for example, as biocatalysts in enzymatic interesterification, transesterification, esterification, alcoholysis, amidation, transamidation and/or hydrolysis reactions, where the final products of said reactions are essentially free of coloration and/or contamination such as silicon compounds and/or any monomer or oligomer desorbed from the enzyme polymer support.

The purified lipase preparations according to the present disclosure exhibit equivalent or higher catalytic activity in enzymatic processing and re-forming of oils and fats, including of interesterification of oils and fats and transesterification of oil glycerides and an alkyl alcohol for production of partial glycerides, fatty acids alkyl esters and glycerol at any predetermined ratio.

Further disclosed herein is a process of enzymatically producing omega-3 fatty acids concentrates comprising enzymatic transesterification omega-3 containing oils, that may be derived from fish, plant and/or oleaginous microorganisms with a short-chain alkyl alcohol in the presence of a purified immobilized lipase as disclosed herein or prepared by the purification methods disclosed herein, where the omega-3 fatty acids concentrates obtained have low color intensity (optionally presented by OD) and low levels of other contaminants such as silicon compounds and/or any monomer or oligomer desorbed from the enzyme polymer support.

Further disclosed is a process of producing re-formed interesterified oils and fats having low color intensity (optionally presented by OD), low content of contaminants such as silicon compounds, and low content, essentially absence of any monomer/s or oligomer/s desorbed from the enzyme polymer support, by any one of enzymatic interesterification, transesterification, esterification, alcoholysis, amidation, transamidation and/or hydrolysis reaction, using a purified immobilized lipase preparation disclosed herein or prepared by the purification methods disclosed herein.

Disclosed herein are purified lipase preparations, comprising a lipase, the lipase being immobilized on a macroporous insoluble support (as described herein), characterized in that the preparations are essentially devoid of residual contaminants present in the fermentation broth of said fermentation process such as pigments, chromophores and other color-producing substances, as well as silicon derivatives and others, which contaminants are adsorbed onto the said solid support during the immobilization of the lipase; and also essentially free of other contaminants that may emanate from the supporting polymer/polymeric resin, such as monomers or oligomers, specifically short oligomers.

### BRIEF DESCRIPTION OF THE FIGURES

In order to better understand the subject matter that is disclosed herein and to exemplify how it may be carried out in practice, embodiments will now be described, by way of non-limiting example only, with reference to the accompanying drawings, in which:

FIG. 1 shows the color of Lipase QLM buffer solution at different enzyme concentrations (0.0%, 0.1%, 0.5%, 1%, 2%, and 3%).

FIG. 2 shows the color of Lipase QLM buffer solution at different enzyme concentrations (0.0%, 0.1%, 0.5%, 1%, 2%, and 3%) after addition of equivalent volume of soybean oil on top of the aqueous enzyme solutions.

FIG. 3 shows the color of Lipase QLM solution at different enzyme concentrations (0.0%, 0.1%, 0.5%, 1%, 2%, and 3%) after addition of equivalent volume of soybean oil on top of the aqueous enzyme solutions, mixing vigorously for two hours at 30° C. and then centrifugation for phase separation.

FIG. 4 shows the color of Lipase QLM solution at different enzyme concentrations (0.0%, 0.1%, 0.5%, 1%, 2%, and 3%) after addition of equivalent volume of soybean oil on top of the aqueous enzyme solutions, mixing vigorously for 24 hours at 30° C. and then centrifugation for phase separation.

FIG. 5 The OD at 420 nm for the oil medium after 24 hours of mixing of 10% w/w of Lipase QLM immobilized on different commercial polymeric resins (see Table 1) in fish oil without the addition of water. Mixing rate was 170 rpm at a temperature of 30° C.

FIG. 6 The OD at 420 nm for the oil medium after 24 hours of mixing of 10% w/w of Lipase QLM immobilized on different commercial polymer resins (see Table 1) in fish oil containing 3% w/w of sodium bicarbonate solution. Mixing rate was 170 rpm at a temperature of 30° C.

FIG. 7 The transesterification activity of Lipase QLM immobilized on different polymer resins (for abbreviations see Table 1) after 3 hours of reaction between fish oil and ethanol using the same batch of biocatalyst in 20 consecutive cycles. For reaction conditions see Table 11.

FIG. 8 Transesterification of fish oil with ethanol to produce FAEEs using the same batch of biocatalyst in 20 consecutive batches. Reaction conditions: fish oil (10 g), 0.3 ml of 0.1M sodium bicarbonate solution and lipase immobilized on either MMA cross-linked with DVB or cross-linked DVB polymer resin (1 g) were mixed vigorously. Ethanol (at molar ratio between ethanol and oil of 2:1) was added stepwise in three equivalent batches each one hour apart. The reaction medium was shaken at 170 rpm and 30° C.

FIG. 9 The total fish oil FAEEs and omega-3 FAEEs in the transesterification reaction medium using the same batch of Lipase QLM immobilized on MMA cross-linked with DVB polymer resin in 20 consecutive batches. Reaction conditions: See FIG. 4.

FIG. 10 The total fish oil FAEEs and omega-3 FAEEs in the transesterification reaction medium using the same batch of Lipase QLM immobilized on cross-linked phenol-formaldehyde polycondensate polymer resin in 20 consecutive cycles. Reaction conditions: See FIG. 4.

FIG. 11 FTIR Spectra for Lipase QLM immobilized on a macroporous resin composed of polymethyl methacrylate crosslinked with DVB at different stages of the transesterification reaction of fish oil with ethanol. (1) before reaction, (2) after one hour reaction of batch 1, (3) after one hour reaction of batch 1 and then washing the biocatalyst with n-hexane followed by drying, (4) after 6 hours reaction using the same batch of biocatalyst in five consecutive batches, (5) after 6 hours reaction using the same batch of biocatalyst in five consecutive batches and then washing the biocatalyst with n-hexane followed by drying.

FIG. 12 FTIR Spectra for the Lipase QLM immobilized on a macroporous resin composed of polymethyl methacrylate crosslinked with DVB at different stages of the transesterification reaction of fish oil with ethanol. (1) before reaction, (2) after 6 hours reaction of batch 1 and then washing the biocatalyst with n-hexane followed by drying, (3) after 6 hours reaction using the same batch of biocatalyst in three consecutive batches and then washing the biocatalyst with n-hexane followed by drying, (4) after 6 hours reaction using the same batch of biocatalyst in five consecutive batches and then washing the biocatalyst with n-hexane followed by drying, (5) after 6 hours reaction using the same batch of biocatalyst in 10 consecutive batches and then washing the biocatalyst with n-hexane followed by drying.

### DETAILED DESCRIPTION OF EMBODIMENTS

Disclosed herein are methods for the preparation of purified immobilized lipase preparations essentially using alcoholysis reactions. Generally, a lipase immobilized on an organic or inorganic support is brought into contact with a fatty acid source such as an oil, containing water or aqueous alkaline buffer solution to form a reaction medium with adjusted suitable pH, to which a suitable alkyl alcohol is added, and the alcoholysis reaction is carried at a suitable temperature to form mainly fatty acid alkyl esters. When a major part of the glycerides and free fatty acids present in the oil fat source, specifically an oil, have been converted to fatty acid alkyl esters with free glycerol formed as a byproduct, the reaction is stopped by removing the immobilized enzyme preparation from the reaction medium, which can be optionally washed with a suitable solvent, for example n-hexane. The same batch of enzyme is again used in the same reaction with fresh reactants, for several cycles of reaction, and is isolated at a suitable stage of the reaction. The repeated use of the immobilized enzyme preparation removes pigments and other impurities, to yield a highly pure enzyme preparation that is essentially devoid of pigments and other impurities emanating from the fermentation process that was used for preparing the enzyme itself, and/or from the immobilization step or the immobilizing support.

A significant advantage of the disclosed method of purification of immobilized enzymes is that low-cost oils may be used. The fatty acid alkyl esters resulting from the alcoholysis reaction can be used, for example as biodiesel. This strategy is cost effective, as the purified immobilized lipase preparation can subsequently be used for processing expensive oils such as, for example, fish and oleaginous oils, to yield oils enriched with n-3 fatty acids such as DHA and EPA, particularly for alimentary, nutraceutical, pharmaceutical and cosmetic products that are subject to strict regulatory standards.

A major concern for use of lipases in their native as well as immobilized forms remains the deteriorated quality of the final treated product due to the presence of impurities, in particular, coloring agents (stains and pigment components), organo-silicon derivatives normally added to reduce foam in the fermentation processes used to manufacture the enzymes, and specific polymer resin support constituents, such as monomers or oligomers. It is one of the objects of the present disclosure to provide a new method for the preparation of purified lipases in their immobilized form, so as to reduce their content of coloring components and other contaminants normally present in fermentation broths such as silicon derivatives, which may desorb/leach from the lipase preparations into the oil/fat reaction medium and cause deterioration of product quality.

Thus, in general, the present disclosure provides methods for purifying a lipase preparation comprising at least one reaction cycle comprising the steps of (1) reacting a reaction substrate comprising at least one fatty acid source comprising oils, glycerides, free fatty acids and/or fatty acid alkyl esters (FAAE) with an alkyl alcohol, in the presence of at least about 100 ppm water or aqueous alkaline buffer solution and up to about 70% w/w, and a lipase preparation in crude or immobilized form, to yield fatty acid alkyl esters and glycerol, allowing the reaction to proceed until at least part of the reaction substrate, for example at least 10%, 20%, 30%, 40% or 50% of the reaction substrate is converted to FAAE and (2) collecting the medium by filtration to obtain the immobilized purified enzyme, and optionally repeating said reaction cycle 1-10 times, each reaction cycle using a fresh reaction substrate in step (1), using the same batch of enzyme used in the first cycle for each of said repeated reaction cycles.

In all aspect and embodiments of the invention disclosed herein, the level of water or aqueous alkaline buffer solution in the reaction medium is from 100 ppm to 70% w/w, for example, about 0.5% w/w, 1% w/w, 2% w/w, 3% w/w, 4% w/w, 5% w/w, 10% w/w and up to 70% w/w, and any sub-ranges or specific ranges in between such as 05.1% w/w, 1-2% w/w, 1-3% w/w, 5-7%, 5-10%.

More specifically, disclosed herein is a method for purifying a lipase preparation comprising at least two consecutive reaction cycles, each reaction cycle comprising the steps of:
(a) providing a reaction substrate comprising at least one fatty acid source, specifically at least one of oils, fats, glycerides, free fatty acids and fatty acid alkyl esters;
(b) adding to said reaction substrate water or aqueous alkaline buffer at more than 100 ppm up to 70% to form a reaction medium;
(c) providing an immobilized lipase preparation comprising a lipase immobilized on an organic or inorganic support;
(d) adding the said lipase preparation to said reaction medium to form a reaction mixture, wherein the pH of the reaction medium is a pH of 4-11 and wherein the temperature of the reaction mixture is 10-50° C.;
(e) subjecting said fatty acid source to alcoholysis by stepwise adding to said reaction mixture an alkyl alcohol, at a molar ratio of at least 2:1 between said alcohol and said reaction substrate, and stiffing or shaking or recirculating the resulting mixture until at least part of said reaction substrate, optionally at least 10%, 20%, 30%, 40% or 50% of said reaction substrate are converted to fatty acid alkyl esters;
(f) adding alkyl alcohol to the reaction mixture of step (e) to reach a total molar ratio of 3:1, specifically where support is a hydrophobic and/or mild hydrophobic polymer resin, between said alcohol and said reaction substrate, and allowing the reaction to proceed until conversion of said reaction substrate to fatty acid alkyl esters exceeds 90%;
(g) collecting the reaction medium of step (f) by filtration and keeping the immobilized lipase preparation, and optionally washing the immobilized lipase preparation with a suitable solvent, such as but not limited to n-hexane; where following filtration, the reaction medium filtrate separates into two phases, an upper (lighter) organic/oil phase comprising the formed fatty acids alkyl esters preferably at more than 50%, free fatty acids and mono-, di- and residual tri-glycerides preferably at less than 10%, and a lower (heavier) phase comprising the formed glycerol and water, wherein the upper organic/oil phase comprises extracted hydrophobic pigment components and other hydrophobic contaminants comprised in the said immobilized lipase preparation, the lower phase comprises extracted hydrophilic pigment components and other hydrophilic contaminants comprised in the said immobilized lipase preparation, and any reaction intermediate products including free fatty acids, fatty acids soaps, mono- and di-glycerides comprise extracted amphiphilic pigment components and other amphiphilic contaminant comprised in the said immobilized lipase preparation; and measuring the color intensity in said upper oil phase by suitable means, wherein the color intensity (optionally expressed by optical density (OD)) of the said oil phase is lower than a solution of said lipase;
(h) repeating at least once, optionally 1-10 times, said reaction cycle comprising said steps (a)-(g), using in each cycle the same batch of the immobilized lipase preparation provided in step (c) of the first reaction cycle, with fresh reaction substrate and alkyl alcohol in each cycle, until the color intensity (optionally expressed as optical density (OD)) of the oil phase of said filtrate is reduced to a desired level as compared to the color intensity of the oil phase obtained in said first reaction cycle, yielding an immobilized lipase of higher purity and of at least comparable enzymatic activity to that of the immobilized lipase preparation provided in step (c) of the first reaction cycle.

In specific aspects and embodiments, the presently disclosed method employs alcoholysis reactions for producing purified immobilized lipases, the method comprising the following steps:
(a) bringing a lipase immobilized on organic or inorganic support into contact with a substrate that is a triglyceride oil, the oil containing more than about 100 ppm and up to 70%, specifically, for example, from about 0.2% to about 7% water or an alkaline buffer solution to form a reaction mixture, such that the pH of the mixture is in the range of from about 4 to about 11, and the mixture is maintained at a temperature of about 10-50° C.; optionally adding to the mixture jointly or separately at least one of tri-, di- and monoglycerides, free fatty acids, and fatty acids alkyl esters as additional substrate/s for an alcoholysis reaction between said substrate/s and an alcohol;
(b) stepwise adding to the mixture prepared in (a) an alkyl alcohol at a molar ratio of at least 2:1 of alcohol: oil triglycerides and stiffing magnetically, mechanically by propeller, by reaction medium recirculation or by shaking, so as to initiate enzymatic alcoholysis reaction between said oil and oil components said glycerides, said free fatty acids, and/or said fatty acid alkyl esters and said alcohol, which can be straight-chain or branched-chain alkyl alcohol, of short, medium and long alkyl chain. In some specific embodiments of the presently disclosed method ethanol is used as the alcohol;
(c) when the reaction reaches more than 50% conversion of the substrate/s to fatty acids alkyl esters, an additional amount of alcohol is added, specifically when the support is a hydrophobic or mild hydrophobic polymeric resin support, to reach a total ratio of alcohol: oil triglycerides on molar basis of 3:1, so as to reach conversion of more than 90% of the substrate/s to fatty acids alkyl esters, with mono- and di-glycerides and glycerol as byproduct;
(d) the reaction medium is collected by filtration and the immobilized enzyme is isolated and the same batch of isolated immobilized enzyme is repeatedly used in repeated steps (a)-(d), in at least one additional consecutive cycle, or 2, 3, 4, and up to 10 or more repeated cycles of alcoholysis; following filtration, the reaction medium filtrate separates, either spontaneously or by centrifugation into two phases, an “upper” (lighter) oil phase comprising the fatty acids alkyl esters formed, preferably at more than 70% and free fatty acids, mono-, di- and residual tri-glycerides preferably at less than 10%, and a “lower” (heavier) phase comprising the formed glycerol and water; the upper oil phase extracts the hydrophobic pigments components and other hydrophobic contaminants adsorbed onto the immobilized lipase preparation, while the lower phase extracts the hydrophilic pigments components and other hydrophilic contaminants adsorbed on the immobilized lipase. The reaction products intermediates, namely, free fatty acids, fatty acids soaps, mono- and di-glycerides extract amphiphilic pigments components and other amphiphilic contaminant adsorbed on the immobilized enzyme;
(e) repeating steps a-d, each repeat (cycle) with the immobilized lipase isolated at end of the preceding reaction cycle, until the color intensity of the oil phase is reduced to the desired level as compared to the color intensity of the starting material, or otherwise determined.

It is noted that the above recited level of water or aqueous alkaline buffer solution, such as from about 100 ppm to about 70% of weight of the fatty acid source, specifically oil as basis, is exemplary. The upper level of water or the aqueous alkaline buffer solution mixed with the fatty acid source and immobilized biocatalyst used in the methods of preparing purified immobilized lipase preparations and in the various production processes disclosed herein that use of the purified lipase preparations, can reach much higher levels of, for example, 10%, 15%, 20%, 25%, 30%, 35% and up to about 70% of weight of the fatty acid source, specifically oil as basis. The recited level of water or aqueous alkaline buffer solution includes any water residual confined in the immobilized biocatalyst preparation, as apparent from the description of immobilization of commercially available enzymes lipases presented below.

The immobilized enzyme preparation isolated after removal of the reaction medium is an immobilized lipase of higher purity compared to the preparation before treatment, with transesterification/interesterification/esterification/amidation/transamidation/hydrolysis activity at least comparable to the activity before purification, namely the same lipase immobilized on the same support, without being used without in the above reaction steps (a)-(e), in one or more reaction cycles, and so is its stability.

The purification method of the present disclosure thus provides stable and active purified immobilized lipases that can be to be used as biocatalysts in enzymatic interesterification, transesterification, esterification, amidation, transamidation, and hydrolysis, or any combination of thereof, with minor effect of coloration and contamination of the final reaction product/s.

Commercially available lipases may be provided in solid and liquid forms, i.e., diluted or dissolved in water optionally containing a polyol (such as glycerol and ethylene glycol), or in a buffer solution of pH adjusted to the range of 4-11 and then immobilized on a suitable support. Specific pH ranges suitable for use in all aspects and embodiments of the present invention may be, but are not limited to 7.0, 7.5, 8.0 or 8.5. The water/buffer solution is removed from the wet support comprising the adsorbed lipase and other contaminants and is then lyophilized to reach a water content less than 50% by weight, and preferable less than 10%. The same can be applied when using powder lipase preparation which first is solubilized in water/buffer solution and then follow the same aforementioned procedure to yield the immobilized lipase. The resulting immobilized lipase preparations usually contain residual amounts of confined water, as mentioned above. These immobilized lipase preparations are specific non-limiting examples of immobilized lipase preparation to be purified by the process/es of the present disclosure.

Specific commercially available lipases that can be used purified by methods of the present disclosure Commercially available lipases that can be used purified by methods of the present disclosure and then used in industrial production process as disclosed herein include, but are not limited to Lipozyme RM, Palatase, Lipozyme CALB-L, Lipozyme CALA-L, Lipozyme TL 100L, and Eversa Transform (all from Novozymes, Denmark); Lipase QLM, Lipase SL, Lipase TL and Lipase PL (all from Meito Sangyo, Japan); and Lipase PS, Lipase BD, and Lipase AK (all from Amano Enzymes, Japan).

Other lipases used herein are derived from Rhizomucor miehei, Pseudomonas sp., Pseudomonas cepacia, Pseudomonas fluorescens, Rhizopus niveus, Mucor javanicus, Rhizopus oryzae, Aspergillus niger, Penicillium camembertii, Burkholderia ubonensis (strain PL266-QLM) and cepacia, Alcaligenes sp., Acromobacter sp., Burkholderia sp., Thermomyces lanuginosa, Humicola lanuginosus, Chromobacterium viscosum, Candida antarctica B, Hyphozyma sp., Candida parapsilosis, Candida rugosa, Candida antarctica A, H. insolens, Crytococcus spp., Geotricum candidum Pseudomonas (Burkholderia) cepacia, Pseudomonas stutzeri or papaya seeds, and pancreatin Immobilization of lipases used in the present disclosure is described in Example 1.

Fatty acid sources used in all aspects and embodiments of the methods of purification and processes for production disclosed can be an oil, such as a plant oil, including but not limited to soybean oil, canola oil, rapeseed oil, olive oil, MCT oil, castor oil, palm oil, sunflower oil, safflower oil, peanut oil, cotton seed oil, Jatropha oil, coconut oil or corn oil; algal oil; fish oil; oleaginous microorganisms derived oil; waste cooking oil; and any mixtures of at least two thereof; a fat, such as but not limited to animal-derived fat or brown grease; free fatty acids, that can be saturated or unsaturated fatty acids of 12-20 carbon atoms, including mono- or polyunsaturated fatty acids (as used herein, fatty acids having four or more double bonds in their carbon chain) and short and medium-chain fatty acids of 2-12 carbon atoms; glycerides, including mono-, di- and triglycerides of short-, medium- and long-chain fatty acids and their mixtures at any ratio; and fatty acid alkyl esters, including but not limited to methyl, ethyl or longer alkyl fatty acid esters, wax esters and sterol esters, and any mixture of at least two of said oil, fat, free fatty acids, glycerides, fatty acid alkyl esters, phospholipids such as, but not limited to lecithin and glycolipids.

In all aspects and embodiments of the methods of purification and processes for production disclosed herein an alcohol can be short-chain alkyl alcohol comprised of 1-6 carbon atoms (specifically, but not limited to ethanol and methanol), medium-chain alkyl alcohol comprised of 8-12 carbon atoms, or long-chain alkyl alcohol comprised of 14-22 carbon atoms. Alcohols used herein can also be polyalcohols, such as ethylene and propylene glycol, and glycerol.

In all aspects and embodiments of the purification methods and production methods disclosed herein, where oils and/or fats are used as the fatty acid source, the final reaction products are comprised of fatty acids alkyl esters, preferably higher than 50%, 70%, 75%, 80%, 85% and 90% w/w, residual triglycerides at lower than 10% w/w, and free fatty acids and partial glycerides (mono- and di-glycerides) at lower than 10% w/w, and glycerol as byproduct of the transesterification reaction.

In other aspects and embodiments of the purification methods and production methods disclosed herein, free fatty acids are used as the fatty acid source, under similar or identical reaction conditions as when oil serves as fatty acid source as described above, the esterification reaction products are fatty acids alkyl esters, with water as byproduct.

In other aspects and embodiments of the purification methods and production methods disclosed herein, fatty acids alkyl esters can be used as the fatty acid source under similar or identical reaction conditions as when oil serves as fatty acid source as described above, and the reaction products are “new” fatty acids alkyl esters with an alcohol as a byproduct of the alcoholysis reaction. By “new” fatty acid alkyl esters is meant that the alkyl moieties of the starting fatty alkyl ester are replaced by the alkyl moiety/moieties of the alcohol/s used.

In other aspects and embodiments of the purification methods and production methods disclosed herein, a mixture of free fatty acids and triglycerides, at any ratio, can be used as the fatty acid source under similar or identical reaction conditions as when oil serves as fatty acid source as described above, and the reaction products are fatty acids alkyl esters, with glycerol and water as byproducts of the esterification/transesterification reactions.

In yet other aspects and embodiments of the purification methods and production methods disclosed herein, a mixture of fatty acids and glycerides (mono-, di-, and tri-glycerides), at any ratio, can be used as the fatty acid source under similar or identical reaction conditions as when oil serves as fatty acid source as described above, and the reaction products are fatty acids alkyl esters with glycerol and water as byproducts of the esterification/transesterification reactions.

In further other aspects and embodiments of the purification methods and production methods disclosed herein, glycerides (mono-, di-, and tri-glycerides) in pure form or in combination at any ratio, can be used as the fatty acid source under similar or identical reaction conditions as when oil serves as fatty acid source as described above, and the reaction products are fatty acids alkyl esters, with glycerol as byproducts of the transesterification reactions catalyzed by the immobilized lipase.

In further other aspects and embodiments of the purification methods and production methods disclosed herein, said fatty acid source is a mixture of triglycerides, free fatty acids and lecithin gum, at any ratio, with similar or identical reaction conditions as when oil serves as fatty acid source as described above, and the esterification/transesterification reaction products are fatty acids alkyl esters, with glycerol, water, lyso-phospholipids and glycerophospholipids as byproducts. In these specific aspects and embodiments of the present disclosure the biocatalyst is particularly a phospholipase.

The methods disclosed herein for purification of immobilized lipases and phospholipases can be applied to lipases and phospholipases immobilized on various supports, which may be hydrophobic, mild hydrophobic, hydrophilic or mixed hydrophobic/hydrophilic support. Thus, the support can be an anion- or cation-exchange resin, a hydrophilic organic polymer, such as, but not limited to polyacrylate, polymethyl methacrylate, polymethyl methacrylate or cross-linked phenol formaldehyde condensate, or hydrophobic organic polymer such as, but not limited to polyvinyl alcohol, polydivinylbenzene and polystyrene, and any mixture thereof. The support can be a mixed hydrophobic/hydrophilic support, such as, but not limited to a support comprising divinylbenzene (DVB) and methylmethacrylate (MMA) units. The “mixed” hydrophobic/hydrophilic polymers are also referred to herein as “mild” hydrophobic polymers. The enzymes to be purified can also be immobilized on an inorganic support such as, but not limited to silica, Celite, diatomaceous earth and perlite. A “mild” hydrophobic support, as used herein, may comprise different, for example alternating, domains of hydrophilic and hydrophobic monomer/s, for example hydrophilic domains of one or two hydrophilic monomers and hydrophobic domains of one or two hydrophobic monomers. The ratio between hydrophilic and hydrophobic domains may be from 1:10 to 10:1, for example 1:1, 1:2, 1:3, 1:4, 1:5, 1:6, 1:7, 1:8, 1:9, and 1:10, or any ratio in between. In preferred embodiments, a mild hydrophobic polymeric support comprises about 30-60% w/w hydrophobic domains and from 70-40%, respectively, hydrophilic domains. An example of an MMA crosslinked with DVB polymer, is presented in Example 13 and other examples below. Specific mild hydrophobic resin used herein the commercially available resin comprised of MMA crosslinked with DVB (sold under Lewatit^{R} VP OC1600, manufactured by LanXess, Germany)

The immobilized lipase preparations can be in powder form, for example with particle size of 1-100 microns, or in the form of beads, typically of 0.01-2 mm diameter.

The lipases purified by the disclosed purification methods or production processes of the present disclosure can be random or position-specific, particularly lipases with sn-1,3 positional specificity. Further, the lipases may or may not have a selectivity toward to certain type of fatty acids, such as short-, medium- and long-chain fatty acids, saturated, mono-unsaturated or polyunsaturated fatty acids.

The color intensity of the reaction medium can be quantified as optical density measured at various wavelengths, which may be adjusted according to specific contaminants desorbed into the reaction product, and other parameters.

In particular aspects and embodiments disclosed herein, the color intensity of the reaction medium is measured as optical density at wavelength of 420 nm, which was chosen as an indication for desorption of pigments/silicon compounds/monomers or oligomers from the resin polymer/s used as a carrier for the immobilized enzyme, into the reaction medium, when the immobilized enzyme is used as a biocatalyst. It has been shown that different commercial enzyme preparations at various concentrations of aqueous or organic enzyme solutions after removal of non-solubles, dispose linear absorbance at wavelength of 420 nm. It is for this reason that in the present work absorbance of the oil reaction medium and/or water phases after their separation and removal of the biocatalyst at 420 nm was chosen to serve as a measure for the amount of pigment components and other contaminants desorbed/leached from the biocatalyst used either in its crude liquid/solid or immobilized forms, into the treated reaction medium. Samples of the reaction mixtures were taken periodically, centrifuged at 10,000 rpm and then the separated oil phase was mixed with a similar volume of iso-propanol in order to dilute the pigment concentration to maintain it within the linear absorbance region at 420 nm.

Thus, disclosed herein are purified immobilized lipase preparations characterized in that the optical density of the product of a reaction between a fatty acid source and an alcohol in a reaction medium containing water or alkaline buffer in the presence of said purified lipase has an optical density that is reduced compared to optical absorbance of the product of the same reaction carried out in the presence of an identical immobilized lipase preparation that is not purified.

As shown in the following examples, the presently disclosed method of purification of immobilized lipase preparations is superior to just mixing the immobilized lipase preparation with an oil and repeating mixing step, as detailed above, and presented in the Examples and the Figures.

The optical density of the product of a reaction between a fatty acid source and an alcohol in a reaction medium containing water or alkaline buffer in the presence of a purified lipase preparation as disclosed herein and/or obtained by the purification methods disclosed herein is reduced compared to optical density of the product of the same reaction carried out in the presence of an identical immobilized lipase preparation that is not purified.

Specific non-limiting examples of purified lipase preparations disclosed herein are preparation of Rhizomucor miehei, Pseudomonas sp., Pseudomonas cepacia, Pseudomonas fluorescens, Burkholderia ubonensis (strain PL266-QLM, also referred to herein as “Lipase QLM”) and cepacia, Alcaligenes sp., Burkholderia sp., Thermomyces lanuginosa, Humicola lanuginosus, Candida antarctica B, Hyphozyma sp., Candida parapsilosis, Candida antarctica A, Pseudomonas (Burkholderia) cepacia and Pseudomonas stutzeri, immobilized on a suitable hydrophobic linear or branched aromatic or hydrophobic aliphatic polymer-based support. More specifically, the lipase can be immobilized on a macroporous resin polymer that is a hydrophobic polymer, a mild hydrophobic or a mixed hydrophobic/hydrophilic polymer.

A specific example is a purified Lipase QLM immobilized on macroporous methylmethacrylate beads, where the optical absorbance at 420 nm of the oil phase of the product of soybean oil treated by one reaction 24 hours cycle with ethanol at an oil to ethanol molar ratio of 1:3 in the presence of said immobilized lipase at a concentration of 10% w/w is 0.150 OD, and 0.100 OD after three reaction cycles each of 24 hours using the same batch of said immobilized lipase, compared to optical density of 0.750 OD and 0.360 OD for soybean oil mixed with 10% w/w immobilized lipase QLM preparation for a first cycle of 24 hours and for three cycles of 24 hours each with same batch of immobilized lipase, and 0.054 OD for soybean oil alone. When used with MCT oil, the optical absorbance at 420 nm of the oil phase of the product of MCT oil treated by one reaction cycle of 24 hours with ethanol at an oil to ethanol molar ratio of 1:3, in the presence of said immobilized lipase at a concentration of 10% w/w is 0.150 OD and 0.050 OD after three reaction cycles each 24 hours when using the same batch of immobilized enzyme, compared to 0.530 OD and 0.082 OD for MCT oil mixed with 10% w/w immobilized lipase QLM for a first cycle of 24 hours and for three cycles each of 24 hours and each with the same batch of immobilized lipase, and 0.013 OD for MCT oil alone. When used with fish oil, the optical absorbance at 420 nm of the oil phase of the product of fish oil treated by one reaction cycle of 24 hours with ethanol at an oil to ethanol molar ratio of 1:3 in the presence of said immobilized lipase at a concentration of 10% w/w is 0.7 OD and 0.32 OD after three reaction cycles each 24 hours when using the same batch of immobilized enzyme, compared to 0.86 OD and 0.5 OD for fish oil mixed with 10% w/w immobilized lipase for a first 24 hours cycles and for three 24 hours cycles each with the same batch of immobilized lipase, and 0.216 OD for pure fish oil.

Purified lipases disclosed herein can be used in a variety of synthetic production processes, specifically industrial-scale processes, yielding products that are of low, if any, color intensity and low, if any, content of contaminants that are present in currently used immobilized biocatalysts preparations, such as silicon derived contaminants. Such processes can be, but are not limited to transesterification of oil glycerides and an alcohol for production of partial glycerides, fatty acids alkyl esters and glycerol at any predetermined ratio, with a low color intensity and free of contaminants, or transesterification of oil triglycerides and an alcohol for production of partial glycerides (mono- and di-glycerides), fatty acids alkyl esters and glycerol, with a low color intensity (optionally expressed as optical density (OD)) and free of contaminants and “leachables”.

Thus, the purified immobilized lipase preparations disclosed herein have advantages in catalyzing reactions of, for example, omega-3 fatty acid concentrates with low color intensity and low content of other contaminants that are present in immobilized biocatalysts, as described above. The present disclosure therefore provides a process for producing n-3 fatty acid concentrates by transesterification of omega-3 oils derived from fish, plant and/or oleaginous microorganisms with a short-chain alcohol, using a purified immobilized lipase according to the present disclosure, wherein the produced n-3 fatty acid concentrates have a low color intensity and low content of said contaminants.

Essentially, the presently disclosed methods of purifying immobilized lipase preparations by various enzymatic reactions using oils and fats as substrates, provide for an optical density (color intensity) of the reacted oil or fat that is substantially similar to that of the starting oil or fat, following at least one reaction cycle. The present purification methods also provide for removal of other contaminants as described herein from the immobilized lipase preparations, as shown, for example, by their FTIR spectra.

In more detail, the present disclosure further provides a method for reducing levels of pigment components and color intensity, as well as level of other contaminants such as, for example, silicon-derived contaminants that desorb from conventional non-purified immobilized lipase preparations and other specific polymer resin support constituents, such as monomers or oligomers, using the purified immobilized lipase preparations of the present disclosure for the transesterification of oils and fats when reacted with different alkyl alcohols, typically methanol and ethanol, for the preparation of pure or mixtures of glycerides (mono-, di- and tri-glycerides), in which processes the formed fatty acids alkyl esters are distilled off at the end of the reaction to end up with a mixture of glycerides which can be then post-treated to yield pure glycerides (mono-, di- and tri-glycerides). A non-limiting example of this application is the selective transesterification of fish and other omega-3 fatty acids containing oils for the removal of non-omega-3 fatty acids (such as saturated, mono-, di- and tri-unsaturated fatty acids) from the glycerol backbone of the oil glycerides as fatty acids ethyl esters, which are totally or partially distilled off, ending up with a mixture of dominantly glycerides enriched with omega-3 fatty acids. This mixture can be further fractionated to yield separately and dominantly, mono-, di- or tri-glycerides mixtures enriched with omega-3 fatty acids.

Still further, the present disclosure further provides a method for reducing levels of pigment components and color intensity, as well as level of other contaminants such as, for example, silicon-derived contaminants that desorb from conventional non-purified immobilized lipase preparations and other specific polymer resin support constituents, such as but not limited to, using the purified immobilized lipase preparations of the present disclosure for partial hydrolysis of omega-3 containing oils and fats for the preparation of pure or mixtures of glycerides (mono-, di- and tri-glycerides) where produced saturated, mono-, di- and tri-unsaturated free fatty acids are distilled off at the end of the reaction, to end up with a mixture of glycerides enriched with omega-3 fatty acids which can be further post-treated to obtain pure glycerides. An example of this application but not limited is the partial hydrolysis of fish and oleaginous oil using a lipase with low selectivity toward omega-3 fatty acids, where the formed free fatty acids at the end of reaction are distilled off and ending up with a mixture of glycerides enriched with omega-3 fatty acids which can be post-treated further to yield pure mono-, di-glycerides, or can be re-esterified to form triglycerides

In other aspects and embodiments, the present disclosure provides processes for producing re-formed (re-structured) interesterified fats and oils with low color intensity and low contamination, using a purified immobilized lipase according to the present disclosure or prepared by a purification method according to the present disclosure. Non-limiting examples for restructured fats and oils include the following:
a. Acidolysis or interesterification reaction of oil/fat triglycerides with free fatty acids or fatty acids alkyl esters, respectively, such as production of OPO by substituting the fatty acyl group on the sn-1 and sn-3 of palm oil mid-fraction with oleic acid or oleic acid ethyl ester and maintaining the palmitic acid on the sn-2 position unchanged,
b. Production of cocoa butter equivalent from palm oil by substituting the oleic acid at the sn-1 and sn-3 positions mainly with stearic and palmitic acids and maintaining oleic acid at the second position,
c. Production of oil spreads by interesterification of palm oil stearin and soybean or canola oils in order to obtain a desired SFI (Solid Fat Index).

Still further, the present disclosure further provides a method for reducing levels of pigment components and color intensity, as well as level of other contaminants such as, for example, silicon-derived contaminants that desorb from conventional non-purified immobilized lipase preparations and other specific polymer resin support constituents, such as monomers or oligomers, using the purified immobilized lipase preparations of the present disclosure for partial hydrolysis of oils and fats, in the preparation of pure or mixtures of glycerides (mono-, di- and tri-glycerides), in which processes produced free fatty are distilled off at the end of the reaction to end up with a mixture of glycerides, which can be further post-treated to obtain yield glycerides. A non-limiting example of this application is the partial hydrolysis of oils and fats, where at the end of reaction the free fatty acids formed are distilled off, ending up with a mixture of glycerides which can be further post-treated to yield pure mono-, di- glycerides, or they can be re-esterified to form triglycerides.

In a further aspect, the present disclosure further provides a method for reducing levels of pigment components and color intensity, as well as level of other contaminants such as, for example, silicon-derived contaminants that desorb from conventional non-purified immobilized lipase preparations and other specific polymer resin support constituents such as monomers or oligomers, using the purified immobilized lipase preparations of the present disclosure for the esterification of free fatty acids and different alkyl alcohols or polyalcohols, for the preparation of esters and pure or mixtures of glycerides (mono-, di- and tri-glycerides) where free fatty acids or fatty acids alkyl esters are distilled off at the end of the reaction to end up with esters or a mixture of glycerides which can be post-treated further to obtain pure glycerides (mono-, di- and tri-glycerides). An example of this application but not limited is the esterification of free fatty acids with a long-chain alcohol or with glycerol where free fatty acids, unreacted alcohols, and generated water at the end of the reaction are distilled off and ending up with a mixture of esters or glycerides which can be purified further to produce pure esters or glycerides.

The present disclosure further provides a method for reducing levels of pigment components and color intensity, as well as level of other contaminants such as, for example, silicon-derived contaminants that desorb from conventional non-purified immobilized lipase preparations and other specific polymer resin support constituents, such as monomers or oligomers, using the purified immobilized lipase preparations of the present disclosure for the transesterification of fatty acids short-chain alkyl esters and medium- or long-chain alkyl alcohols, for the preparation of fatty acids medium- or long-chain alkyl esters where the unreacted fatty acids short-chain alkyl esters are distilled off at the end of the reaction to end up with a residue of enriched fatty acids medium- or long-chain alkyl esters. A non-limiting example of this application is the selective immobilized lipase-catalyzed transesterification of free fatty acid ethyl esters of fish oil with a medium- or long-chain alcohol, using a purified immobilized lipase with low selectivity toward omega-3 fatty acids, such that any unreacted omega-3 fatty acids ethyl esters are distilled off from the reaction mixture, to yield a concentrate of omega-3 fatty acids ethyl esters in the distillate and fatty acids medium- and/or long-chain alkyl esters in the residue.

In specific aspect and embodiments, the present disclosure further provides a method for reducing levels of pigment components and color intensity, as well as level of other contaminants such as, for example, silicon-derived contaminants that desorb/leach from conventional non-purified immobilized lipase preparations and other specific polymer resin support constituents, such as monomers or oligomers, using the purified immobilized lipase preparations of the present disclosure for interesterification of two or more different oils and fats, for the preparation of inter-esterified homogenous oils/fats mixture for food industry, for example margarine industry. A non-limiting example of this application is the interesterification of palm oil with a canola oil (e.g., a mixture of 50%:50%), to obtain an interesterified oil that serves as a base for the industrial production of margarines and various oil-based or oil-containing spreads.

In yet further specific aspect and embodiments, the present disclosure further provides a method for reducing levels of pigment components and color intensity, as well as level of other contaminants such as, for example, silicon-derived contaminants that desorb/leach from conventional non-purified immobilized lipase preparations and other specific polymer resin support constituents, such as monomers or oligomers, using the purified immobilized lipase preparations of the present disclosure for the acidolysis of oil/fat triglycerides with a specific free fatty acid or fatty acid short-chain alkyl esters (saturated, unsaturated, or polyunsaturated, short- or medium-chain fatty acid) where the formed free fatty acids or fatty acids short-chain alkyl esters are distilled off at the end of the reaction to end up with a modified oil/fat enrich a specific fatty acid at the sn-1,3 positions, or at random distribution on the glycerol backbone of glycerides. A non-limiting example of this application is the acidolysis reaction between palm oil mid-fraction enriched with palmitic acid at the sn-2 position and oleic acid or oleic acid ethyl esters in the presence of a purified immobilized lipase of the present disclosure with an sn-1,3 specificity to yield a mixture of modified fat/oil enriched with palmitic acid at the sn-2 position and oleic acid at the sn-1 and 3 positions, and free fatty acids or fatty acids ethyl esters. Free fatty acids or their fatty acids ethyl esters are distilled off from the reaction mixture to yield a modified, re-structured oil/fat which can be used as human milk-fat substitute.

In other specific aspect and embodiments, the present disclosure further provides a method for reducing levels of pigment components and color intensity, as well as level of other contaminants such as, for example, monomers or oligomers may leach from the polymer resin used for immobilization of the enzyme, silicon-derived contaminants that desorb/leach from conventional immobilized lipase preparations, such as Novozym 435, Lipozyme RM IM and others, using the purified immobilized lipase preparations of the present disclosure for the acidolysis of oil/fat triglycerides with a specific free fatty acid or fatty acid short-chain alkyl esters (saturated, unsaturated, or polyunsaturated, short- or medium-chain fatty acid) where the formed free fatty acids or fatty acids short-chain alkyl esters are distilled off at the end of the reaction to end up with a modified oil/fat enrich a specific fatty acid at the sn-1,3 positions, or at random distribution on the glycerol backbone of glycerides. A non-limiting example of this application is the acidolysis reaction between palm oil mid-fraction enriched with palmitic acid at the sn-2 position and oleic acid or oleic acid ethyl esters in the presence of a purified immobilized lipase of the present disclosure with an sn-1,3 specificity to yield a mixture of modified fat/oil enriched with palmitic acid at the sn-2 position and oleic acid at the sn-1 and 3 positions, and free fatty acids or fatty acids ethyl esters. Free fatty acids or their fatty acids ethyl esters are distilled off from the reaction mixture to yield a modified, re-structured oil/fat which can be used as human milk-fat substitute.

### Definitions

The term “lipase” as used herein also encompasses “phospholipase”. Generally, lipase as used herein refers to a naturally occurring lipase enzyme obtained from a natural source by industrial fermentation process/es. The terms “lipase” and “phospholipase” are also referred to herein as “the enzyme” or the “biocatalyst”.

Further, the term “lipase” as used herein encompasses both “crude” or “free” or “non-immobilized” lipase, and a lipase immobilized on a support as described herein, which may be referred to as “immobilized lipase”. Both crude and immobilized lipase may be referred to herein as “lipase preparation”. It is to be understood that a “commercially available lipase” encompasses lipases derived from different strains of microorganisms, suspended in excipients, such as, for example, lactose or cyclodextrins, to produce the solid form of a lipase preparation, or a lipase dissolved in water optionally containing a polyol, such as glycerol, ethylene or propylene glycol, or salt such as sodium chloride to produce a liquid lipase preparation.

The term “support” is to be taken to mean a solid matrix or polymer or polymeric resin on which the biocatalyst is immobilized by either physical bonding or chemical bonding. The terms “support”, “matrix”, “supporting polymer” “polymeric resin” may be used herein interchangingly. A “mixed” hydrophobic/hydrophilic polymer recited herein is also referred to herein as “mild” hydrophobic.

By “oligomer” as used herein is meant any fragment of a polymer/polymeric resin comprising from two to about 8 or 10 monomers, such as dimers, trimers, tetramers, etc.

The term “comparable enzymatic activity” as used herein is to be taken to mean enzymatic activity of the purified immobilized lipase preparation that is at least 90%, 95%, 100%, 110% or more compared to that of a corresponding non-purified immobilized lipase preparation. “A corresponding non-purified immobilized lipase preparation” is an immobilized preparation of the same lipase, on the same support, initially immobilized by the same immobilization method that has not been used as biocatalyst in any catalyzed reaction.

The terms “oil phase”, “organic phase”, “top phase” and “upper phase” when referring to a phase of the reaction medium or filtered reaction medium at any stage of the reaction, as described herein, may be used interchangingly. Similarly, the terms “lower phase”, “bottom phase” and “hydrophilic phase” when referring to a phase of the reaction medium or filtered reaction medium, at any stage of the reaction, may be used interchangingly.

The terms “optical density (OD)” and “optical absorption” may be used herein interchangingly. As described, color intensity of any reactant or reaction product as described herein is measured and exhibited by optical density. Therefore, the term “color intensity” can also be used synonymously with “optical density (OD)”.

As used in this specification and the appended claims, the singular forms “a”, “an” and “the” include plural referents unless the content clearly dictates otherwise. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The terms “enzyme selectivity” and “enzyme specificity” are used herein interchangingly.

As used herein, the term “about” is meant to encompass deviation of ±10% from the specifically mentioned value of a parameter, such as temperature, pressure, concentration, yield, concentration, etc.

Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals therebetween. It should be noted that where various embodiments are described by using a given range, the range is given as such merely for convenience and brevity and should not be construed as an inflexible limitation on the scope of the invention. Accordingly, the description of a range should be considered to have specifically disclosed all the possible sub-ranges as well as individual numerical values within that range.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present disclosure to its fullest extent. The following preferred specific embodiments are, therefore, to be construed as merely illustrative, and not limitative of the claimed invention in any way.

### Example 1

### Lipase Immobilization Method

Lipases were immobilized following standard procedures where liquid or powder lipase preparation derived from a certain microorganism is solubilized in buffer solution of 0.1M at a certain pH value, for example 7.5. An organic or inorganic polymer resin was introduced into the lipase solution. The mixture was shaken at room temperature for 8 hours. The mixture was filtered, and the immobilized enzyme beads were dried to reduce the water content to less than 10%. Different resins were used including polymer resins based on aromatic (polystyrene/divinylbenzene) and aliphatic monomers (methyl methacrylate) or any of their combinations, and crosslinked phenol-formaldehyde polycondensate, to obtain resins of hydrophobic characteristics, mild hydrophilic and hydrophilic resins. The commercial names (registered trademarks or trade names owned by proprietor) of the resin polymers used in this study are listed in Table 1.

TABLE 1
The commercial names, manufacturers and compositions of
polymer resins used for the immobilization of lipases

| | Commercial | | |
|---|---|---|---|
| | name of | | |
| Designation | polymer resin | Manufacturer | Composition |
| PSL 1 | Seplite LSF | Sunresin, China | Styrene/divinylbenzene copolymer |
| | 903 | | macroporous adsorbent resin |
| PSL 2 | Lewatit VP OC | Lanxess, | Polymetheylmethacrylate/divinylbenzene |
| | 1600 | Germany | copolymer macroporous adsorbent resin |
| PSL 3 | Amberlite | DuPont, USA | Macroporous, weak base anion resin. |
| | FPA66 | | Styrene-divinylbenzene Copolymer |
| PSL 4 | Amberlite | DuPont, USA | Macroporous adsorbent resin crosslinked |
| | FpX66 | | aromatic polymer |
| PSL 5 | Amberlite SD-2 | DuPont, USA | Macroporous adsorbent resin - Styrene- |
| | | | divinylbenzene |
| PSL 6 | Amberlite | DuPont, USA | Macroporous adsorbent resin- crosslinked |
| | XAD16HP | | divinylbenzene |
| PSL 7 | Amberlite XAD | DuPont, USA | Macroporous adsorbent resin - crosslinked |
| | 16N | | divinylbenzene |
| PSL 8 | Amberlite XAD | DuPont, USA | Adsorbent is a macroporous, non-ionic, |
| | 7HP | | crosslinked aliphatic polymer |
| PSL 9 | Amberlite XAD | DuPont, USA | Macroporous adsorbent resin- crosslinked |
| | 1180N | | divinylbenzene |
| PSL 10 | Duolite A568 | DuPont, USA | Weak base anion exchange resin - |
| | | | Crosslinked phenol-formaldehyde |
| | | | polycondensate (hydrophilic resin). |
| PSL 11 | Amberlite XAD | DuPont, USA | Macroporous adsorbent resin- crosslinked |
| | 1600N | | divinylbenzene |

Crude lipases and immobilized lipases were used to catalyze the transesterification/esterification of oils and fats using refined, bleached, and deodorized (RBD) soybean, medium-chain triglycerides (MCT) or fish oil (18:12) and ethanol as an alcohol donor. The optical density (OD) of the reaction medium in the upper oil phase as well as in the lower water/glycerol phase was measured by a spectrophotometer (UV-Visible Spectrophotometer, Thermo Scientific, Evolution 260 B 10 Model) at a wavelength of 420nm after appropriate mixing of the samples with n-hexane or iso-propanol for the upper phase and with water for lower phase.

### Example 2

### Transfer of Pigments and Other Contaminants from Powder Lipase Preparation to Oil

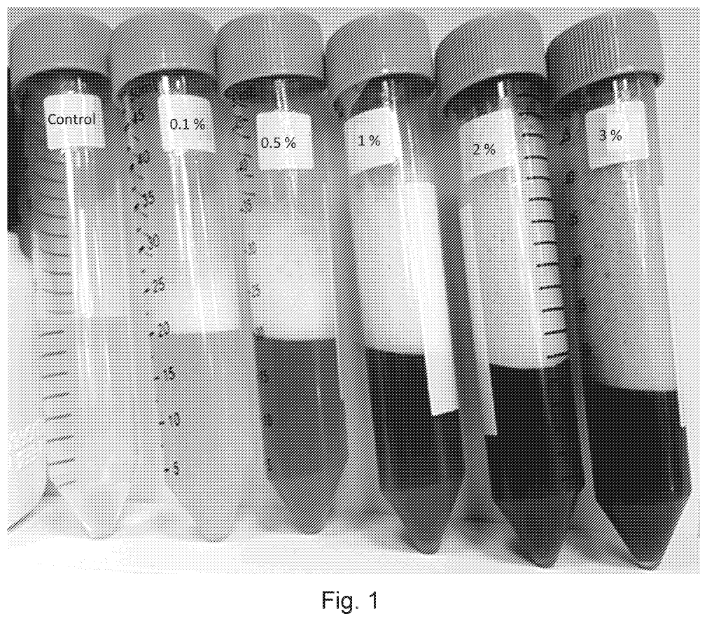

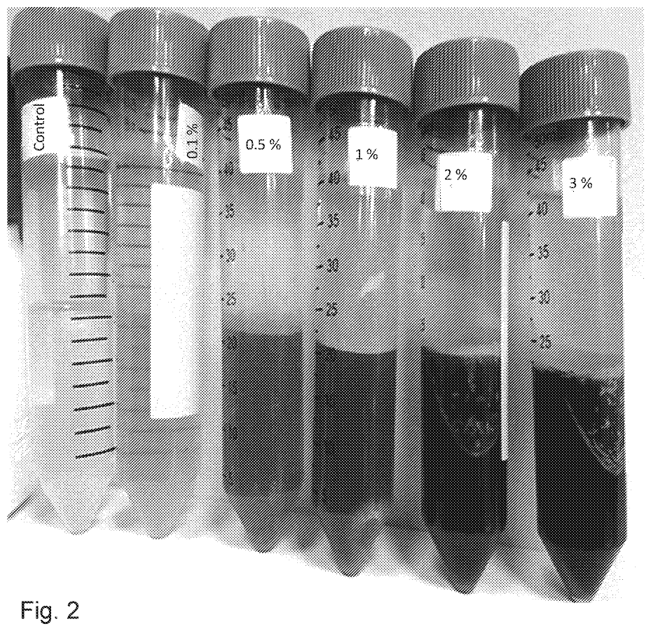

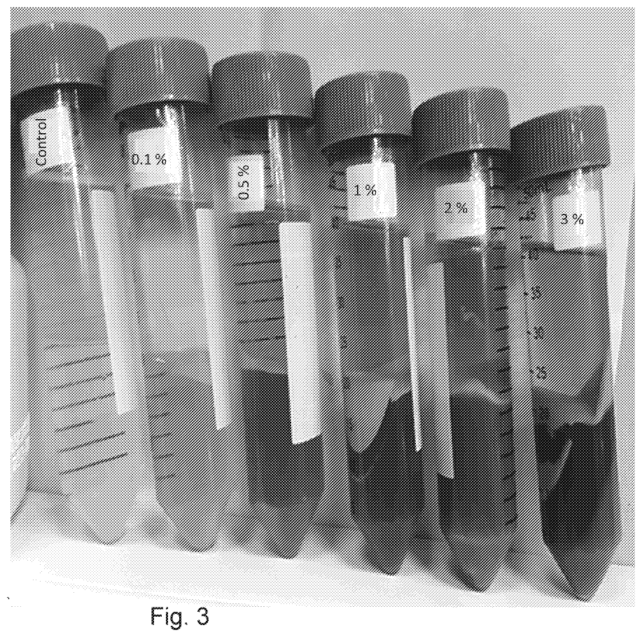

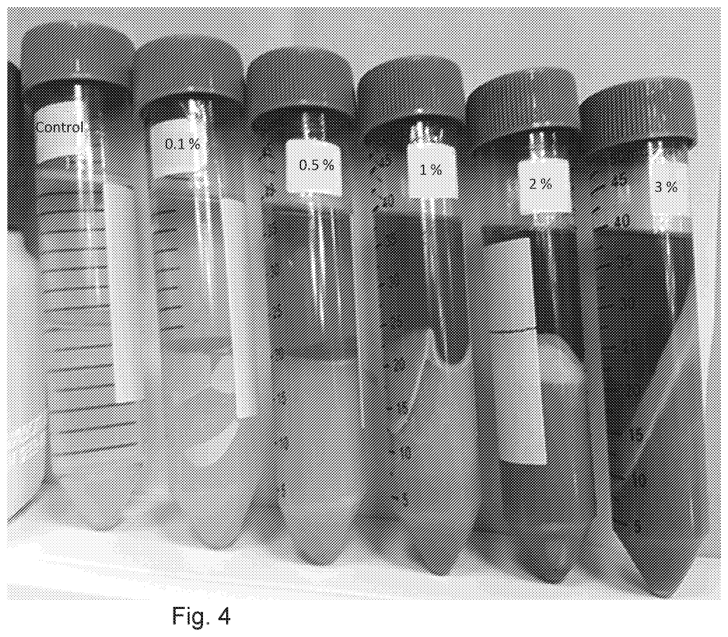

Different concentrations (0.1%, 0.5%, 1%, 2% and 3% based on weight of soybean oil) of commercially available Lipase QLM (Meito Sangyo, Japan) were prepared by mixing of the enzyme powder at 170 rpm in refined soybean oil at 30° C. FIGS. 1-4 show visually the extent of color of the treated oil with time when oil was treated with enzyme preparation at 30° C. Table 2 shows the optical density (OD) at wavelength of 420 nm for soybean oil after mixing with different concentrations of crude Lipase QLM in soybean oil at different time intervals at 30° C. The results presented in Table 2 show that pigment ingredients and other contaminants migrate from the crude powder enzyme preparation to the oil phase. The results show that the rate of transfer of pigments components from the powderous enzyme preparation to the oil medium is time and concentration of enzyme dependent.

TABLE 2
The optical density (OD) at wavelength of 420 nm for soybean
oil after mixing at 30° C. with different concentrations
of Lipase QLM in soybean oil at different time intervals.

| Crude Lipase | OD (420 nm) | | | | |
|---|---|---|---|---|---|
| QLM (wt. %) | 0 h | 2 h | 4 h | 6 h | 24 h |
| 0 | 0.054 | 0.054 | 0.054 | 0.054 | 0.054 |
| 0.1 | 0.076 | 0.079 | 0.082 | 0.083 | 0.107 |
| 0.5 | 0.156 | 0.168 | 0.186 | 0.198 | 0.209 |
| 1 | 0.211 | 0.249 | 0.288 | 0.302 | 0.317 |
| 2 | 0.299 | 0.382 | 0.496 | 0.499 | 0.552 |
| 3 | 0.365 | 0.489 | 0.655 | 0.652 | 0.737 |

### Example 3

### Transfer of Pigments and Other Contaminants from Aqueous Lipase Solution Phase to Oil Phase

Different concentrations (0.1%, 0.5%, 1%, 2% and 3% based on weight of buffer solution) of commercially available Lipase QLM (Meito Sangyo, Japan) were prepared by vigorous mixing of the enzyme powder in phosphate buffer solution of pH 7 at room temperature.

After complete solubilization of the enzyme, an equivalent volume of soybean oil was added on top of the enzyme aqueous solution (FIG. 1). Both phases have been mixed at 170 rpm for 24hours at 30° C. Samples after centrifugation of the mixtures were taken periodically from the top oil phase and mixed with equivalent volumes of iso-propanol, centrifuged at 10000 rpm and then absorbance at 420 nm was measured. FIGS. 1-4 visually show the extent of color in each phase at different times of mixing. Table 3 shows the optical density (OD) at 420 nm wavelength for the soybean oil phase after mixing with different concentrations of Lipase QLM dissolved in aqueous buffer solution at different time intervals.

The results presented in FIGS. 1-4 and Table 3 show clearly that pigment components and other contaminants present in the aqueous crude enzyme preparation of Lipase QLM can pass from the aqueous phase to the oil phase and contaminate the oil phase with different ingredients. Also, the results presented in Table 3 show that the pigment components can pass from the enzyme aqueous solution to the oil phase on a linear basis up to a certain degree with time.

TABLE 3
The optical density (OD) at 420 nm wavelength for the
soybean oil phase after mixing at 30° C. with
different concentrations of Lipase QLM dissolved in
aqueous buffer solution at different time intervals.

| Crude Lipase | OD (420 nm) | | | | |
|---|---|---|---|---|---|
| QLM (wt. %) | 0 h | 2 h | 3 h | 6 h | 24 h |
| 0 | 0.054 | 0.054 | 0.054 | 0.054 | 0.054 |
| 0.1 | 0.054 | 0.059 | 0.06 | 0.063 | 0.069 |
| 0.5 | 0.055 | 0.069 | 0.083 | 0.092 | 0.12 |
| 1 | 0.078 | 0.079 | 0.098 | 0.112 | 0.192 |
| 2 | 0.098 | 0.099 | 0.122 | 0.149 | 0.317 |
| 3 | 0.109 | 0.123 | 0.162 | 0.198 | 0.332 |

### Example 4

### Purification Process of Immobilized Lipases

Purification of immobilized lipases was carried out by using the biocatalyst to catalyze transesterification reaction of soybean oil and ethanol to form fatty acids ethyl esters, partial glycerides and glycerol as byproducts of the reaction. Also immobilized lipase purification was carried out, by using the biocatalyst to catalyze esterification reaction of oleic acid and ethanol to form fatty acid ethyl esters, and water as byproduct of the reaction. Furthermore, purification of immobilized lipases was carried out by using the biocatalyst to catalyze simultaneous esterification and transesterification reactions of a mixture of soybean oil and oleic acid at different ratios of ethanol to form fatty acid ethyl esters, partial glycerides, and glycerol and water as byproducts of the reaction. The purified immobilized lipase after removal of the reaction medium was used to catalyze other types of reactions without desorption/transfer of pigment components or other contaminants into the treated oil medium, as well as the immobilized enzyme exhibits higher activity than the same immobilized enzyme however without applying the purification method.

Purification process/Transesterification reaction conditions: Refined soybean oil (20 g) containing 2% wt. of sodium bicarbonate solution of 0.1M and different concentrations (1%, 5%, 10%, 15% and 20% based on weight of oil) of Lipase QLM (Meito Sangyo, Japan) immobilized on a macroporous methyl methacrylate (MMA) polymer beads of 200-1000 microns in diameter, were mixed vigorously for 20 min Ethanol (at a ratio between ethanol and oil of 3:1 on molar basis) was added stepwise in three equivalent batches each one hour apart. The reaction medium containing the immobilized lipase preparation was shaken at 170 rpm and 30° C. Samples were withdrawn from the reaction mixture at different time intervals for determination of the enzyme activity as well as for determining of color intensity as an indication for presence of contaminants After 24 hours of reaction, the reaction medium was filtered off and a new fresh batch of substrates was introduced using the same batch of immobilized enzyme. The conversion to fatty acid ethyl esters reached 30-80% after 6 hours of reaction, and after 24 hours reached 80-95%, depending on the amount of biocatalyst. Ten consecutive reaction cycles were carried out using the same batch of immobilized lipase. Table 4 shows the color intensity in the organic/oil phase at different time intervals in 10 cycles for the transesterification reaction medium (top organic phase) after 24 h reaction. The results show that under the proposed reaction conditions, pigment ingredients and other components adsorbed on the polymer support undergo extraction into the top organic/oil phase as well as into the glycerol/water phase after the first reaction cycle and a purified immobilized enzyme is obtained, that can be used in different applications. Repeating the runs using the same batch of immobilized enzyme would totally remove the pigment components and other contaminants from the immobilized enzyme preparation.

TABLE 4
The optical density (OD) of the oil phase at 420 nm at
different time intervals using the same batch of immobilized
enzyme in 10 consecutive cycles. Reaction conditions:
Soybean oil (20 g), 0.4 ml sodium bicarbonate solution
of 0.1M and different amounts (1%, 5%, 10%, 15% and 20%
based on oil weight) of immobilized Lipase QLM were shaken
at 170 rpm and at 30° C. Ethanol at a molar ratio
to oil of 3:1 was added to the reaction mixture in three
equivalent batches, each one hour apart.

| Immobilized | OD (420 nm) | | | | |
|---|---|---|---|---|---|
| lipase (%) | 0 h | 2 h | 4 h | 6 h | 24 h |
| Batch 1 | | | | | |
| 0% | 0.054 | 0.054 | 0.054 | 0.054 | 0.054 |
| 1% | 0.058 | 0.092 | 0.099 | 0.105 | 0.106 |
| 5% | 0.061 | 0.092 | 0.091 | 0.094 | 0.096 |
| 10% | 0.065 | 0.096 | 0.095 | 0.099 | 0.15 |
| 15% | 0.08 | 0.124 | 0.122 | 0.123 | 0.225 |
| 20% | 0.109 | 0.168 | 0.254 | 0.377 | 0.419 |
| Batch 3 | | | | | |
| 0% | 0.054 | 0.054 | 0.054 | 0.054 | 0.054 |
| 1% | 0.048 | 0.054 | 0.054 | 0.052 | 0.061 |
| 5% | 0.049 | 0.056 | 0.056 | 0.058 | 0.071 |
| 10% | 0.056 | 0.076 | 0.079 | 0.08 | 0.097 |
| 15% | 0.069 | 0.107 | 0.102 | 0.099 | 0.126 |
| 20% | 0.083 | 0.122 | 0.114 | 0.112 | 0.15 |
| Batch 5 | | | | | |
| 0% | 0.054 | 0.054 | 0.054 | 0.054 | 0.054 |
| 1% | 0.045 | 0.049 | 0.062 | 0.057 | 0.065 |
| 5% | 0.05 | 0.068 | 0.066 | 0.066 | 0.077 |
| 10% | 0.054 | 0.084 | 0.086 | 0.083 | 0.113 |
| 15% | 0.066 | 0.111 | 0.11 | 0.107 | 0.148 |
| 20% | 0.073 | 0.131 | 0.125 | 0.131 | 0.191 |
| Immobilized | O.D (420 nm) | | | | |
| lipase (%) | 0 h | 2 h | 4 h | 6 h | 24 h |
| Batch 7 | | | | | |
| 0% | 0.054 | 0.054 | 0.054 | 0.054 | 0.054 |
| 1% | 0.043 | 0.042 | 0.042 | 0.045 | 0.053 |
| 5% | 0.048 | 0.055 | 0.053 | 0.057 | 0.089 |
| 10% | 0.051 | 0.064 | 0.066 | 0.069 | 0.116 |
| 15% | 0.062 | 0.084 | 0.082 | 0.079 | 0.148 |
| 20% | 0.069 | 0.103 | 0.099 | 0.103 | 0.2 |
| Batch 10 | | | | | |
| 0% | 0.054 | 0.054 | 0.054 | 0.054 | 0.054 |
| 1% | 0.049 | 0.048 | 0.054 | 0.048 | 0.052 |
| 5% | 0.053 | 0.052 | 0.052 | 0.054 | 0.062 |
| 10% | 0.056 | 0.054 | 0.056 | 0.058 | 0.065 |
| 15% | 0.054 | 0.055 | 0.058 | 0.068 | 0.071 |
| 20% | 0.058 | 0.064 | 0.072 | 0.071 | 0.077 |

### Example 5

### Purification Process of Immobilized Lipases

Purification of immobilized lipases was carried out by using the biocatalyst to catalyze transesterification reaction of medium-chain triglycerides oil (MCT) and ethanol to form medium-chain fatty acid ethyl esters, partial glycerides and glycerol as byproduct of the reaction. The purified immobilized lipase can be obtained after removal of the reaction medium by filtration.

Purification process/Transesterification reaction conditions: medium-chain triglycerides oil (MCT) (20 g) containing 2% wt. of sodium bicarbonate solution of 0.1M and different concentrations (1%, 5%, 10%, 15% and 20% based on weight of oil) of Lipase QLM (Meito Sangyo, Japan) immobilized on a macroporous MMA polymer beads of 200-1000 microns in diameter, were mixed vigorously for 20 min Ethanol (the ratio between ethanol and the oil is 3:1 in molar basis) was added stepwise in three equivalent batches each one hour apart. The reaction medium containing the immobilized lipase preparation was shaken at 170 rpm and 30° C. Samples were withdrawn from the reaction mixture at different time intervals for determination of enzyme activity as well as for determining of color intensity as an indication for presence of contaminants. After 24 hours of reaction, the reaction medium was filtered off and a new fresh batch of substrates was introduced using the same batch of immobilized enzyme. The reaction conversion to fatty acid ethyl esters reached 30-80% after 6 hours of reaction, and after 24 hours reached 80-95%, depending on the amount of biocatalyst. Ten consecutive reaction cycles were carried out using the same batch of immobilized lipase. Table 5 shows the optical density (OD) of the top organic phase at different time intervals in 5 cycles, reflecting the color intensity.

The results show that under the proposed reaction conditions, pigment ingredients and other components adsorbed on the polymer support undergo extraction into the top organic phase as well as into the bottom glycerol/water phase after the first cycle and obtain the purified immobilized enzyme for different applications. Repeating the runs using the same batch of immobilized enzyme would totally remove the pigment components and other contaminants from the immobilized enzyme.

TABLE 5
The color intensity (as OD) at 420 nm of the oil phase at different
time intervals using the same batch of immobilized enzyme in
10 consecutive cycles. Reaction conditions: MCT oil (20 g),
0.4 ml sodium bicarbonate solution of 0.1M and different amount
(1%, 5%, 10%, 15% and 20% based on oil weight) of immobilized
Lipase QLM were shaken at 170 rpm and at 30° C. Ethanol
at a molar ratio to oil of 3:1 was added in three equivalent
batches into the reaction mixture each one hour apart.

| Immobilized | OD (420 nm) | | | | |
|---|---|---|---|---|---|
| lipase (%) | 0 h | 2 h | 4 h | 6 h | 24 h |
| Batch 1 | | | | | |
| 0% | 0.013 | 0.013 | 0.013 | 0.013 | 0.013 |
| 1% | 0.058 | 0.092 | 0.099 | 0.105 | 0.106 |
| 5% | 0.061 | 0.092 | 0.091 | 0.094 | 0.096 |
| 10% | 0.065 | 0.096 | 0.095 | 0.099 | 0.15 |
| 15% | 0.08 | 0.124 | 0.122 | 0.123 | 0.225 |
| 20% | 0.109 | 0.168 | 0.254 | 0.377 | 0.419 |
| Batch 3 | | | | | |
| 0% | 0.013 | 0.013 | 0.013 | 0.013 | 0.013 |
| 1% | 0.011 | 0.008 | 0.008 | 0.006 | 0.007 |
| 5% | 0.013 | 0.021 | 0.024 | 0.027 | 0.019 |
| 10% | 0.026 | 0.03 | 0.032 | 0.041 | 0.048 |
| 15% | 0.039 | 0.072 | 0.067 | 0.058 | 0.098 |
| 20% | 0.08 | 0.138 | 0.118 | 0.113 | 0.169 |
| Batch 5 | | | | | |
| 0% | 0.013 | 0.013 | 0.013 | 0.013 | 0.013 |
| 1% | 0.01 | 0.009 | 0.008 | 0.008 | 0.008 |
| 5% | 0.011 | 0.013 | 0.019 | 0.023 | 0.008 |
| 10% | 0.019 | 0.018 | 0.016 | 0.015 | 0.009 |
| 15% | 0.023 | 0.022 | 0.021 | 0.021 | 0.015 |
| 20% | 0.031 | 0.031 | 0.036 | 0.041 | 0.053 |

### Comparative Examples

Similar to Examples 4 and 5, further tests for purification of immobilized lipases were carried out using the same biocatalyst and substrates, without addition of an alcohol. Macroporous MMA polymer beads were used as an exemplary support. Results are presented in the following Examples 6 to 11.

### Example 6

A mixture of soybean oil (20 g), 2% wt. of 0.1M sodium bicarbonate solution and different amounts (1%, 5%, 10%, 15% and 20% based on weight of oil) of Lipase QLM immobilized on a macroporous MMA polymer beads of 200-1000 microns in diameter were mixed vigorously for 20 min. The medium containing the immobilized lipase preparation was shaken at 170 rpm and 30° C. Samples were withdrawn from the reaction mixture at different time intervals for determining of the color intensity as an indication for presence of contaminants. After 24 hours of shaking, the reaction medium was filtered off and a new fresh batch of soybean oil was introduced using the same batch of immobilized enzyme. Ten consecutive cycles were carried out using the same batch of immobilized lipase. Table 6 shows the color intensity (as OD) in the organic phase at different time intervals in 10 cycles using the same batch of biocatalyst.

TABLE 6
The color intensity (as OD) of the oil phase at 420 nm
at different time intervals using the same batch of
immobilized enzyme in 10 consecutive cycles. Treatment
conditions: Soybean oil (20 g), 0.4 ml sodium bicarbonate
solution of 0.1M and different amount (1%, 5%, 10%,
15% and 20% based on oil weight) of immobilized Lipase
QLM were shaken at 170 rpm and at 30° C.

| Batch 1 | | | | | |
|---|---|---|---|---|---|
| Immobilized | OD (420 nm) | | | | |
| lipase (%) | 0 h | 2 h | 4 h | 6 h | 24 h |
| 0% | 0.054 | 0.054 | 0.054 | 0.054 | 0.054 |
| 1% | 0.054 | 0.075 | 0.083 | 0.09 | 0.12 |
| 5% | 0.058 | 0.129 | 0.164 | 0.215 | 0.351 |
| 10% | 0.086 | 0.187 | 0.257 | 0.359 | 0.748 |
| 15% | 0.084 | 0.262 | 0.375 | 0.64 | 2.376* |
| 20% | 0.112 | 0.328 | 0.487 | 0.788 | 3.360* |
| Batch 3 | | | | | |
| Immobilized | O.D (420 nm) | | | | |
| lipase (%) | 0 h | 2 h | 4 h | 6 h | 24 h |
| 0% | 0.054 | 0.054 | 0.054 | 0.054 | 0.054 |
| 1% | 0.052 | 0.057 | 0.06 | 0.062 | 0.089 |
| 5% | 0.072 | 0.08 | 0.092 | 0.094 | 0.158 |
| 10% | 0.139 | 0.176 | 0.189 | 0.198 | 0.362 |
| 15% | 0.29 | 0.331 | 0.34 | 0.349 | 0.643 |
| 20% | 0.509 | 0.555 | 0.591 | 0.599 | 1.302** |

| Immobilized | OD (420 nm) | | | | |
|---|---|---|---|---|---|
| lipase (%) | 0 h | 2 h | 4 h | 6 h | 24 h |
| Batch 5 | | | | | |
| 0% | 0.054 | 0.054 | 0.054 | 0.054 | 0.054 |
| 1% | 0.055 | 0.058 | 0.061 | 0.062 | 0.086 |
| 5% | 0.067 | 0.078 | 0.09 | 0.102 | 0.138 |
| 10% | 0.1 | 0.128 | 0.138 | 0.149 | 0.21 |
| 15% | 0.139 | 0.179 | 0.196 | 0.204 | 0.297 |
| 20% | 0.202 | 0.265 | 0.29 | 0.308 | 0.483 |
| Batch 7 | | | | | |
| 0% | 0.054 | 0.054 | 0.054 | 0.054 | 0.054 |
| 1% | 0.056 | 0.057 | 0.06 | 0.064 | 0.085 |
| 5% | 0.06 | 0.068 | 0.072 | 0.079 | 0.105 |
| 10% | 0.076 | 0.095 | 0.104 | 0.113 | 0.148 |
| 15% | 0.101 | 0.124 | 0.139 | 0.151 | 0.234 |
| 20% | 0.159 | 0.198 | 0.217 | 0.235 | 0.344 |
| Batch 10 | | | | | |
| 0% | 0.054 | 0.054 | 0.054 | 0.054 | 0.054 |
| 1% | 0.053 | 0.053 | 0.056 | 0.058 | 0.077 |
| 5% | 0.056 | 0.063 | 0.073 | 0.081 | 0.106 |
| 10% | 0.076 | 0.091 | 0.106 | 0.119 | 0.146 |
| 15% | 0.099 | 0.128 | 0.149 | 0.17 | 0.202 |
| 20% | 0.167 | 0.204 | 0.235 | 0.269 | 0.339 |

*Bold values after 24 hours were normalized with a dilution factor of 4
**Bold values after 24 hours were normalized with a dilution factor of 2

### Example 7

A mixture of MCT oil (20 g), 2% wt. of 0.1M sodium bicarbonate solution and different amounts (1%, 5%, 10%, 15% and 20% based on weight of oil) of Lipase QLM (Meito Sangyo, Japan) immobilized on macroporous MMA polymer beads of 200-1000 microns in diameter, were mixed vigorously for 20 min. The medium containing the immobilized lipase preparation was shaken at 170rpm and 30° C. Samples were withdrawn from the reaction mixture at different time intervals for determining of the color intensity as an indication for presence of contaminants After 24 hours of shaking, the reaction medium was filtered off and a new fresh batch of MCT oil was introduced using the same batch of immobilized enzyme. Ten consecutive cycles were carried out using the same batch of immobilized lipase. Table 7 shows the color intensity in the organic phase (as OD) at different time intervals in 10 cycles using the same batch of biocatalyst.

Throughout the above-mentioned treatment, the immobilized lipase preparation undergoes only partial removal of color components and other adsorbed organic components to form an insufficiently purified immobilized lipase which still leaks contaminants even after 24 hours of 10 cycles of wash (Tables 6 and 7).

TABLE 7
The color intensity (as OD) of the oil phase at 420 nm at different
time intervals using the same batch of immobilized enzyme in
10 consecutive cycles. Treatment conditions: MCT oil (20 g),
0.4 ml sodium bicarbonate solution of 0.1M and different amount
(1%, 5%, 10%, 15% and 20% based on oil weight) of immobilized
Lipase QLM were shaken at 170 rpm and at 30° C.

| Immobilized | O.D (420 nm) | | | | |
|---|---|---|---|---|---|
| lipase (%) | 0 h | 2 h | 4 h | 6 h | 24 h |
| Batch 1 | | | | | |
| 0% | 0.013 | 0.013 | 0.013 | 0.013 | 0.013 |
| 1% | 0.005 | 0.013 | 0.014 | 0.013 | 0.012 |
| 5% | 0.018 | 0.054 | 0.067 | 0.071 | 0.118 |
| 10% | 0.05 | 0.143 | 0.169 | 0.193 | 0.525 |
| 15% | 0.219 | 0.605 | 0.763 | 0.922 | 2.436* |
| 20% | 0.342 | 0.719 | 0.879 | 0.988 | 3.636* |
| Batch 3 | | | | | |
| 0% | 0.013 | 0.013 | 0.013 | 0.013 | 0.013 |
| 1% | 0.01 | 0.012 | 0.012 | 0.011 | 0.011 |
| 5% | 0.02 | 0.021 | 0.023 | 0.026 | 0.037 |
| 10% | 0.036 | 0.042 | 0.044 | 0.047 | 0.082 |
| 15% | 0.291 | 0.615 | 0.641 | 0.675 | 0.706 |
| 20% | 0.463 | 0.899 | 0.891 | 0.906 | 0.998 |
| Batch 5 | | | | | |
| 0% | 0.013 | 0.013 | 0.013 | 0.013 | 0.013 |
| 1% | 0.011 | 0.01 | 0.008 | 0.008 | 0.008 |
| 5% | 0.012 | 0.011 | 0.011 | 0.013 | 0.03 |
| 10% | 0.018 | 0.017 | 0.02 | 0.023 | 0.037 |
| 15% | 0.039 | 0.077 | 0.076 | 0.077 | 0.108 |
| 20% | 0.071 | 0.163 | 0.187 | 0.202 | 0.28 |
| Batch 7 | | | | | |
| 0% | 0.013 | 0.013 | 0.013 | 0.013 | 0.013 |
| 1% | 0.009 | 0.008 | 0.006 | 0.009 | 0.012 |
| 5% | 0.008 | 0.01 | 0.009 | 0.013 | 0.023 |
| 10% | 0.013 | 0.018 | 0.022 | 0.026 | 0.039 |
| 15% | 0.016 | 0.019 | 0.024 | 0.031 | 0.056 |
| 20% | 0.024 | 0.044 | 0.072 | 0.087 | 0.159 |
| Batch 10 | | | | | |
| 0% | 0.013 | 0.013 | 0.013 | 0.013 | 0.013 |
| 1% | 0.011 | 0.01 | 0.011 | 0.01 | 0.013 |
| 5% | 0.011 | 0.008 | 0.009 | 0.011 | 0.03 |
| 10% | 0.018 | 0.026 | 0.028 | 0.033 | 0.07 |
| 15% | 0.021 | 0.044 | 0.045 | 0.048 | 0.131 |
| 20% | 0.043 | 0.01 | 0.103 | 0.111 | 0.223 |

*Bold values after 24 hours were normalized with a dilution factor of 4

### Example 8

A mixture of oleic acid (20 g), 2% wt. of 0.1M sodium bicarbonate solution and different amounts (1%, 5%, 10%, 15% and 20% based on weight of oil) of Lipase QLM (Meito Sangyo, Japan) immobilized on macroporous MMA polymer beads of 200-1000 microns in diameter, were mixed vigorously for 20 min. The medium containing the immobilized lipase preparation was shaken at 170 rpm and 30° C. Samples were withdrawn from the reaction mixture at different time intervals for determining of the color intensity as an indication for presence of contaminants After 24 hours of shaking, the reaction medium was filtered off and a new fresh batch of substrates was introduced using the same batch of immobilized enzyme. Table 8 shows the color intensity in the organic phase at different time intervals in 10 cycles using the same batch of biocatalyst.

Throughout the above-mentioned treatment, the immobilized lipase preparation undergoes partial removal of color components and other adsorbed organic components to form an insufficiently purified immobilized lipase which still leaks contaminants even after 10 cycles each 24 hours.

TABLE 8
The color intensity (as OD) of the oleic acid phase at
420 nm at different time intervals using the same batch
of immobilized enzyme in 10 consecutive cycles. Treatment
conditions: Oleic acid (20 g), 0.4 ml sodium bicarbonate
solution of 0.1M and different amount (1%, 5%, 10%, 15%
and 20% based on oil weight) of immobilized Lipase QLM
were shaken at 170 rpm and at 30° C.

| Batch 1 | | | | | |
|---|---|---|---|---|---|
| Immobilized | OD (420 nm) | | | | |
| lipase (%) | 0 h | 2 h | 4 h | 6 h | 24 h |
| 0% | 0.025 | 0.025 | 0.025 | 0.025 | 0.025 |
| 1% | 0.027 | 0.036 | 0.039 | 0.041 | 0.042 |
| 5% | 0.057 | 0.079 | 0.088 | 0.096 | 0.105 |
| 10% | 0.08 | 0.142 | 0.159 | 0.171 | 0.246 |
| 15% | 0.139 | 0.277 | 0.359 | 0.42 | 0.905 |
| 20% | 0.204 | 0.391 | 0.499 | 0.609 | 1.774* |

| Immobilized | OD (420 nm) | | | | |
|---|---|---|---|---|---|
| lipase (%) | 0 h | 2 h | 4 h | 6 h | 24 hr |
| Batch 3 | | | | | |
| 0% | 0.025 | 0.025 | 0.025 | 0.025 | 0.025 |
| 1% | 0.028 | 0.029 | 0.028 | 0.029 | 0.031 |
| 5% | 0.032 | 0.032 | 0.033 | 0.033 | 0.039 |
| 10% | 0.042 | 0.048 | 0.053 | 0.054 | 0.066 |
| 15% | 0.093 | 0.099 | 0.109 | 0.113 | 0.141 |
| 20% | 0.176 | 0.19 | 0.201 | 0.227 | 0.273 |
| Batch 5 | | | | | |
| 0% | 0.025 | 0.025 | 0.025 | 0.025 | 0.025 |
| 1% | 0.026 | 0.029 | 0.031 | 0.035 | 0.037 |
| 5% | 0.029 | 0.03 | 0.035 | 0.036 | 0.054 |
| 10% | 0.037 | 0.044 | 0.046 | 0.049 | 0.064 |
| 15% | 0.051 | 0.064 | 0.068 | 0.07 | 0.089 |
| 20% | 0.085 | 0.1 | 0.118 | 0.139 | 0.171 |
| Batch 7 | | | | | |
| 0% | 0.025 | 0.025 | 0.025 | 0.025 | 0.025 |
| 1% | 0.023 | 0.025 | 0.024 | 0.024 | 0.025 |
| 5% | 0.025 | 0.026 | 0.027 | 0.029 | 0.034 |
| 10% | 0.031 | 0.037 | 0.04 | 0.044 | 0.061 |
| 15% | 0.045 | 0.053 | 0.054 | 0.059 | 0.091 |
| 20% | 0.05 | 0.063 | 0.066 | 0.071 | 0.129 |
| Batch 10 | | | | | |
| 0% | 0.025 | 0.025 | 0.025 | 0.025 | 0.025 |
| 1% | 0.022 | 0.025 | 0.026 | 0.026 | 0.028 |
| 5% | 0.023 | 0.026 | 0.03 | 0.032 | 0.053 |
| 10% | 0.025 | 0.029 | 0.035 | 0.037 | 0.052 |
| 15% | 0.041 | 0.044 | 0.046 | 0.052 | 0.068 |
| 20% | 0.045 | 0.068 | 0.077 | 0.088 | 0.142 |

*Bold values after 24 hours were normalized with a dilution factor of 2

### Example 9

A mixture of soybean oil and oleic acid with an equivalent weight ratio (20 g), 2% wt. of sodium bicarbonate solution and different amounts (1%, 5%, 10%, 15% and 20% based on weight of oil) of Lipase QLM (Meito Sangyo, Japan) immobilized on macroporous MMA polymer beads of 200-1000 microns in diameter, were mixed vigorously for 20 min. The medium containing the immobilized lipase preparation was shaken at 170 rpm and 30° C. Samples were withdrawn from the reaction mixture at different time intervals for determining of the color intensity (as OD) as an indication for presence of contaminants After 24 hours of shaking, the reaction medium was filtered off and a new fresh batch of soybean oil and oleic acid mixture was introduced using the same batch of immobilized enzyme. Ten consecutive cycles were carried out using the same batch of immobilized lipase. Table 9 shows the color intensity in the organic phase at different time intervals in 10 cycles using the same batch of biocatalyst. Throughout conducting of the above-mentioned treatment, the immobilized lipase preparation undergoes partial removal of color components and other adsorbed organic components to form an insufficiently purified immobilized lipase which still leaks contaminants even after 24 hours of 10 cycles of wash. For example, in Batch 10, after 24 hours, even at a low lipase concentration of 1% w/w, the OD of the oil phase was 0.047, compared to OD of 0.033 of the original soybean oil.

TABLE 9
The color intensity (as OD) at 420 nm of the oil phase
at different time intervals using the same batch of immobilized
enzyme in 10 consecutive cycles. Treatment conditions:
Soybean oil (10 g), oleic acid (10 g), 0.4 ml sodium bicarbonate
solution of 0.1M and different amount (1%, 5%, 10%, 15%
and 20% based on oil weight) of immobilized Lipase QLM
were shaken at 170 rpm and at 30° C.

| Immobilized | OD (420 nm) | | | | |
|---|---|---|---|---|---|
| lipase (%) | 0 h | 2 h | 4 h | 6 h | 24 h |
| Batch 1 | | | | | |
| 0% | 0.033 | 0.033 | 0.033 | 0.033 | 0.033 |
| 1% | 0.035 | 0.061 | 0.072 | 0.079 | 0.127 |
| 5% | 0.039 | 0.101 | 0.118 | 0.15 | 0.274 |
| 10% | 0.045 | 0.143 | 0.187 | 0.219 | 0.602 |
| 15% | 0.078 | 0.225 | 0.239 | 0.344 | 3.032* |
| 20% | 0.108 | 0.302 | 0.338 | 0.411 | 3.444** |
| Batch 3 | | | | | |
| 0% | 0.033 | 0.033 | 0.033 | 0.033 | 0.033 |
| 1% | 0.04 | 0.045 | 0.053 | 0.055 | 0.068 |
| 5% | 0.062 | 0.078 | 0.092 | 0.101 | 0.132 |
| 10% | 0.113 | 0.156 | 0.18 | 0.191 | 0.27 |
| 15% | 0.23 | 0.281 | 0.314 | 0.323 | 0.428 |
| 20% | 0.332 | 0.413 | 0.445 | 0.459 | 0.585 |
| Batch 5 | | | | | |
| 0% | 0.033 | 0.033 | 0.033 | 0.033 | 0.033 |
| 1% | 0.036 | 0.039 | 0.038 | 0.039 | 0.048 |
| 5% | 0.045 | 0.054 | 0.057 | 0.06 | 0.087 |
| 10% | 0.062 | 0.088 | 0.099 | 0.107 | 0.146 |
| 15% | 0.09 | 0.128 | 0.14 | 0.151 | 0.227 |
| 20% | 0.135 | 0.194 | 0.211 | 0.225 | 0.384 |
| Batch 7 | | | | | |
| 0% | 0.033 | 0.033 | 0.033 | 0.033 | 0.033 |
| 1% | 0.034 | 0.039 | 0.045 | 0.045 | 0.054 |
| 5% | 0.039 | 0.045 | 0.05 | 0.054 | 0.062 |
| 10% | 0.054 | 0.065 | 0.072 | 0.088 | 0.121 |
| 15% | 0.072 | 0.095 | 0.104 | 0.113 | 0.163 |
| 20% | 0.106 | 0.133 | 0.151 | 0.167 | 0.239 |
| Batch 10 | | | | | |
| 0% | 0.033 | 0.033 | 0.033 | 0.033 | 0.033 |
| 1% | 0.031 | 0.037 | 0.039 | 0.039 | 0.047 |
| 5% | 0.035 | 0.045 | 0.049 | 0.052 | 0.067 |
| 10% | 0.049 | 0.053 | 0.059 | 0.061 | 0.095 |
| 15% | 0.073 | 0.079 | 0.09 | 0.1 | 0.124 |
| 20% | 0.077 | 0.096 | 0.113 | 0.129 | 0.16 |

**Bold values after 24 hours were normalized with a dilution factor of 4
**Bold values after 24 hours were normalized with a dilution factor of 6

### Example 10

A mixture of MCT oil and oleic acid with an equivalent weight ratio (20 g), 2% wt. of 0.1M sodium bicarbonate solution and different amounts (1%, 5%, 10%, 15% and 20% based on weight of oil) of Lipase QLM (Meito Sangyo, Japan) immobilized on macroporous MMA polymer beads of 200-1000 microns in diameter, were mixed vigorously for 20 min. The medium containing the immobilized lipase preparation was shaken at 170 rpm and 30° C. Samples were withdrawn from the reaction mixture at different time intervals for determining of the color intensity (as OD) as an indication for presence of contaminants. After 24 hours of shaking, the reaction medium was filtered off and a new fresh batch of MCT oil and oleic acid mixture was introduced using the same batch of immobilized enzyme. Ten consecutive cycles were carried out using the same batch of immobilized lipase. Table 10 shows the color intensity of the organic phase at different time intervals in cycles using the same batch of biocatalyst.

Throughout conducting of the above-mentioned treatment, the immobilized lipase preparation undergoes partial removal of color components and other adsorbed organic components to form an insufficiently purified immobilized lipase which still leaks contaminants even after 24 hours of 10 cycles of wash.

TABLE 10
The color intensity (as OD) at 420 nm of the oil phase
at different time intervals using the same batch of immobilized
enzyme in 10 consecutive cycles. Treatment conditions:
MCT oil (10 g), oleic acid (10 g), 0.4 ml sodium bicarbonate
solution of 0.1M and different amount (1%, 5%, 10%, 15%
and 20% based on oil weight) of immobilized Lipase QLM
were shaken at 170 rpm and at 30° C.

| Immobilized | OD (420 nm) | | | | |
|---|---|---|---|---|---|
| lipase (%) | 0 h | 2 h | 4 h | 6 h | 24 h |
| Batch 1 | | | | | |
| 0% | 0.011 | 0.011 | 0.011 | 0.011 | 0.011 |
| 1% | 0.012 | 0.02 | 0.023 | 0.028 | 0.048 |
| 5% | 0.021 | 0.064 | 0.079 | 0.095 | 0.153 |
| 10% | 0.043 | 0.17 | 0.203 | 0.246 | 0.451 |
| 15% | 0.072 | 0.242 | 0.289 | 0.333 | 3.732* |
| 20% | 0.099 | 0.298 | 0.301 | 0.486 | 4.080* |
| Batch 3 | | | | | |
| 0% | 0.011 | 0.011 | 0.011 | 0.011 | 0.011 |
| 1% | 0.013 | 0.015 | 0.019 | 0.023 | 0.023 |
| 5% | 0.023 | 0.031 | 0.042 | 0.048 | 0.069 |
| 10% | 0.069 | 0.074 | 0.082 | 0.09 | 0.135 |
| 15% | 0.179 | 0.201 | 0.225 | 0.247 | 0.308 |
| 20% | 0.352 | 0.384 | 0.415 | 0.432 | 0.557 |
| Batch 5 | | | | | |
| 0% | 0.011 | 0.011 | 0.011 | 0.011 | 0.011 |
| 1% | 0.016 | 0.015 | 0.017 | 0.02 | 0.021 |
| 5% | 0.018 | 0.026 | 0.029 | 0.036 | 0.054 |
| 10% | 0.035 | 0.056 | 0.063 | 0.077 | 0.128 |
| 15% | 0.069 | 0.108 | 0.122 | 0.131 | 0.253 |
| 20% | 0.145 | 0.186 | 0.199 | 0.214 | 0.334 |
| Batch 7 | | | | | |
| 0% | 0.011 | 0.011 | 0.011 | 0.011 | 0.011 |
| 1% | 0.011 | 0.011 | 0.011 | 0.011 | 0.014 |
| 5% | 0.021 | 0.018 | 0.02 | 0.023 | 0.039 |
| 10% | 0.031 | 0.04 | 0.046 | 0.056 | 0.08 |
| 15% | 0.066 | 0.083 | 0.093 | 0.097 | 0.181 |
| 20% | 0.094 | 0.13 | 0.144 | 0.162 | 0.306 |

### Example 11

### Purification Process of Lipase QLM Immobilized on Different Polymer Resins Using Fish Oil (without Supplemented Water) or Fish Oil Containing 3% 0/1M Sodium Bicarbonate Aqueous Solution—Control Experiments

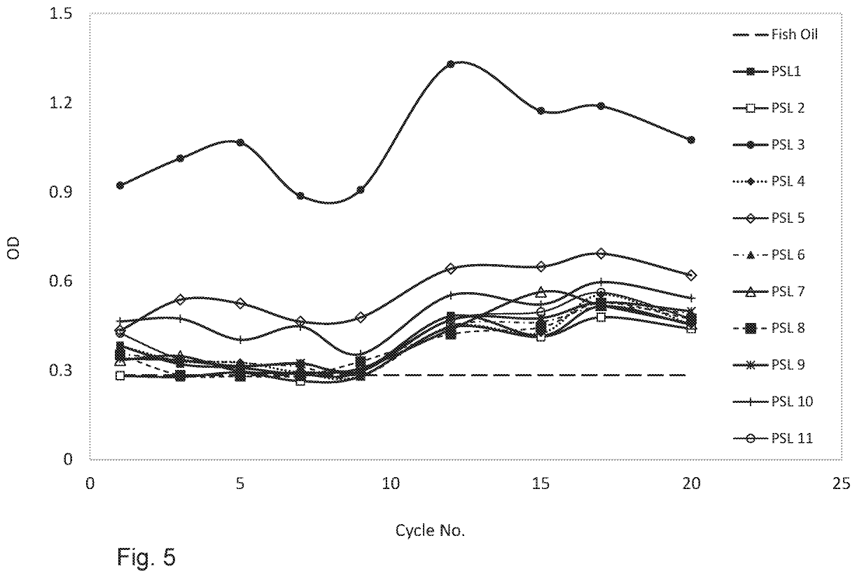

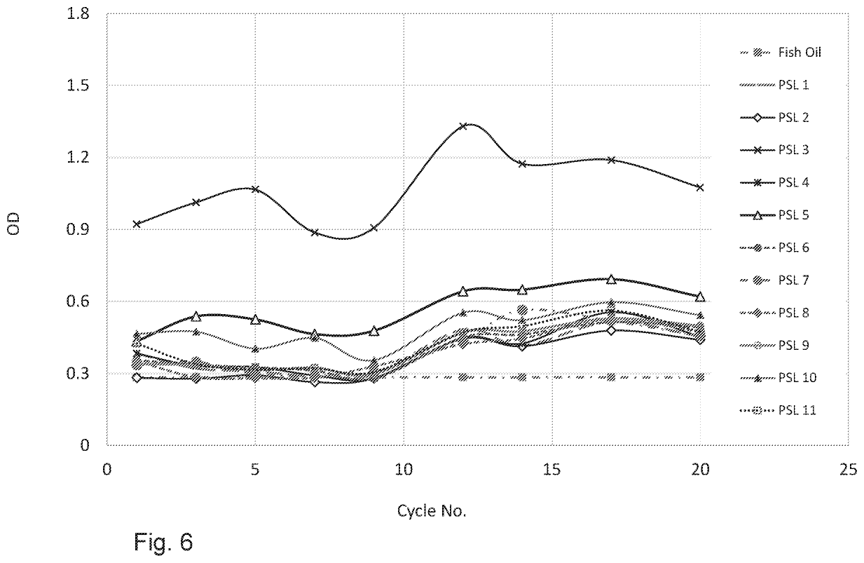

FIGS. 5 and 6 show the OD at 420 nm of the fish oil medium after 24 hours of mixing of Lipase QLM immobilized on different polymer resin supports (10% by weight of oil) with fish oil of low water content (below 200 ppm) or with fish oil containing 3% sodium bicarbonate solution of 0.1M, respectively, at a temperature of 30° C. and mixing at 170 rpm.

The results in FIG. 5 show that the OD after 24 hours of mixing, all fish oil samples which were mixed with the different immobilized lipases, was slightly higher than the OD of fish oil used as a control (with no immobilize lipase) up to Cycle 9, and thereafter increased linearly until Cycle 20. These results indicate that there was a migration of low amounts of contaminants from the immobilized enzyme in the Cycles 1-9, and thereafter (Cycles 10-20) the OD increased linearly and significantly as compared to the OD of the fish oil starting material. The same trend was also observed when aqueous buffer solution of sodium bicarbonate was added to the oil medium (3% by weight), however the OD in Cycles 1-9 as well as up to Cycle 20 was significantly higher than the counterpart experiment conducted without water (FIG. 6). These results indicate that the presence of water leads to significant increment of the rate of release of contaminants from the biocatalyst polymer beads into the oil medium.

### Example 12

### Purification Process of Lipase QLM Immobilized on a Different Polymer Resins Using Fish Oil Containing 3% Sodium Bicarbonate Solution of 0.1M and Ethanol

Tables 11 shows the OD of the upper, organic phase of the reaction medium after 24 hours mixing of 10% by wt. of Lipase QLM immobilized on different polymer supports (See Table 1) with fish oil and ethanol (⅔ on molar basis ethanol/oil) containing 3% sodium bicarbonate solution of 0.1M at a temperature of 30° C. and mixing at 170 rpm. The results in Table 11 show that the OD of the upper phase after 24 hours of reaction was high at the first cycle and decreased linearly during the first 6 consecutive cycles. Table 11 show also that from Cycle 6 and up to Cycle 20 there were no significant changes in the OD as compared to the OD of the control fish oil. These results indicate that regardless of the polymer resin used for the immobilization of the biocatalysts, all polymeric resins released contaminants of hydrophobic characteristics into the oil medium linearly during the reaction Cycles 1-6. The reaction of conversion to fatty acid ethyl esters reached 30-80% after 6 hours of reaction, and after 24 hours reached 80-95%, depending on the type of biocatalyst. The results also show that most of the polymeric resins do not release contaminants to the reaction medium after Cycle 6 and up to Cycle 20, where the OD of the upper oil phase of the reaction medium after 24 hours of reaction was similar to the OD of the fish oil starting material (Control). The same trend was observed when the OD after 24 hours of reaction for the bottom phase of the reaction medium, comprising the byproduct glycerol and the supplemented water, was measured (Table 12). The results show that very high concentrations of contaminants with hydrophilic characteristics were released from all types of immobilized enzyme preparations into the reaction medium and ended up in the bottom phase. The OD of the bottom phase after 6 cycles of using the same batch of biocatalyst did not change significantly indicating no significant release of contaminants from the biocatalysts into the reaction mediums after Cycle 6.

TABLE 11
The OD at 420 nm of the upper oil phase of the reaction medium after 24 hours
mixing of 10% of Lipase QLM immobilized on different resin polymers (PSL-1-
11, See Table 1), by weight of oil with fish oil and ethanol (2/3 on molar basis
ethanol/oil). The reaction medium contained 3% sodium bicarbonate solution
of 0.1M, maintained at temperature of 30° C. and mixing rate of 170 rpm.

| | OD | | | | | | | | | | | |
|---|---|---|---|---|---|---|---|---|---|---|---|---|
| Cycle | Fish | PSL | PSL | PSL | PSL | PSL | PSL | PSL | PSL | PSL | PSL | PSL |
| No. | oil | 1 | 2 | 3 | 4 | 5 | 6 | 7 | 8 | 9 | 10 | 11 |
| 1 | 0.28 | 0.462 | 0.474 | 1.313 | 0.638 | 0.697 | 0.665 | 0.688 | 0.438 | 0.643 | 0.543 | 0.775 |
| 3 | 0.28 | 0.299 | 0.288 | 0.998 | 0.298 | 0.392 | 0.325 | 0.306 | 0.291 | 0.357 | 0.493 | 0.488 |
| 6 | 0.28 | 0.255 | 0.251 | 0.908 | 0.248 | 0.324 | 0.261 | 0.267 | 0.278 | 0.27 | 0.449 | 0.288 |
| 9 | 0.28 | 0.272 | 0.253 | 0.843 | 0.274 | 0.311 | 0.317 | 0.334 | 0.277 | 0.301 | 0.434 | 0.292 |
| 11 | 0.28 | 0.268 | 0.262 | 0.783 | 0.281 | 0.296 | 0.301 | 0.287 | 0.265 | 0.319 | 0.432 | 0.281 |
| 14 | 0.28 | 0.286 | 0.246 | 0.766 | 0.248 | 0.288 | 0.265 | 0.283 | 0.248 | 0.27 | 0.42 | 0.253 |
| 16 | 0.28 | 0.263 | 0.27 | 0.778 | 0.29 | 0.341 | 0.27 | 0.289 | 0.29 | 0.325 | 0.428 | 0.302 |
| 20 | 0.28 | 0.259 | 0.272 | 0.749 | 0.299 | 0.298 | 0.301 | 0.282 | 0.281 | 0.279 | 0.436 | 0.289 |

TABLE 12
The OD at 420 nm of the bottom phase comprising glycerol byproduct and water after 24 hours of reaction.
For reaction conditions see header of Table 11

| Cycle | OD | | | | | | | | | | | |
|---|---|---|---|---|---|---|---|---|---|---|---|---|
| No. | Glycerol | PSL 1 | PSL 2 | PSL 3 | PSL 4 | PSL 5 | PSL 6 | PSL 7 | PSL 8 | PSL 9 | PSL 10 | PSL 11 |
| 1* | 0.01 | 15.76 | 8.56 | 15.76 | 18.9 | 16.8 | 37.52 | 31.78 | 12.6 | 35.4 | 6 | 21.24 |
| 3* | 0.01 | 1.5 | 1.86 | 3.4 | 6.82 | 6.12 | 9.56 | 10.1 | 2.54 | 1.66 | 9.26 | 9.92 |
| 6* | 0.01 | 1.16 | 2.56 | 2.66 | 13.64 | 3.46 | 3.06 | 3.58 | 10.1 | 2.58 | 5.72 | 2.58 |
| 9* | 0.01 | 1.26 | 2.14 | 2.58 | 2.56 | 1.62 | 2.42 | 2.02 | 2.16 | 3.72 | 2.22 | 2.56 |
| 11* | 0.01 | 1.42 | 1.78 | 3.64 | 2.18 | 1.58 | 1.36 | 2.26 | 2.34 | 1.76 | 2.02 | 1.98 |
| 14* | 0.01 | 1.02 | 1.1 | 2.62 | 1.86 | 1.54 | 0.92 | 1.24 | 2.44 | 1.96 | 2.3 | 1.48 |
| 16* | 0.01 | 0.92 | 1.42 | 2.46 | 1.58 | 1.4 | 2.12 | 2.46 | 2.76 | 2.3 | 2.16 | 2.04 |
| 20* | 0.01 | 1.26 | 1.22 | 4.16 | 1.14 | 1.68 | 0.88 | 0.98 | 1.38 | 1.54 | 2.4 | 0.82 |

*When observed OD value was above 1.5, sample was diluted appropriately with water to give OD value below 1.5.
The obtained OD value was multiplied by the dilution factor.

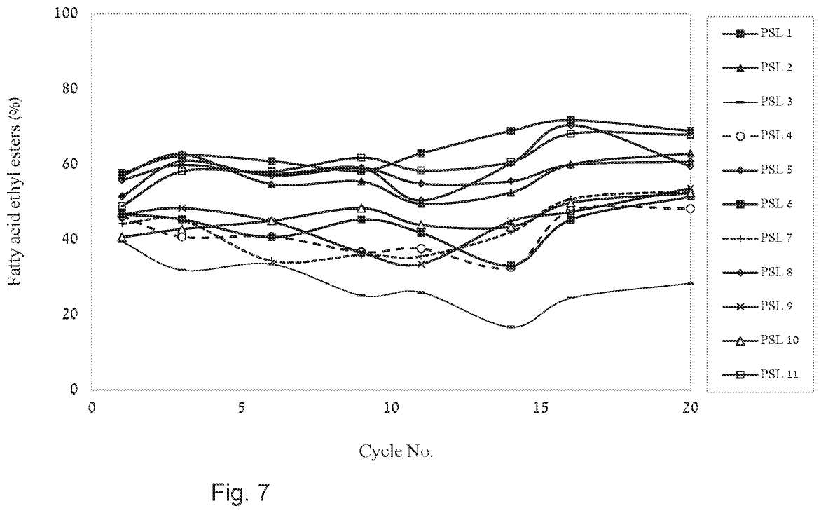

FIG. 7 shows the transesterification activity of the different immobilized lipases after 3 hours of reaction. Unexpectedly, the results show that most of the immobilized lipases even increased their catalytic activity with the number of reaction cycles when the same batch of biocatalyst was reused in 20 consecutive cycles. This result indicates that there was no desorption of the protein enzyme from the polymer resin, while there was selective desorption of contaminants from the surface area of the polymer resins, which leads to better exposure of the immobilized enzyme to its substrates.

### Example 13

### Purification Process of Lipase QLM Immobilized on Different Polymer Resins for Transesterification of Fish Oil in Multiple Uses

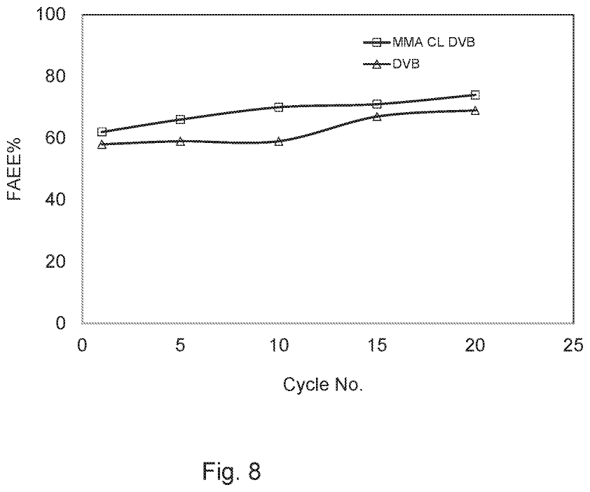

Reaction conditions: A mixture of fish oil (20 g), 3% wt. of 0.1M sodium bicarbonate solution and Lipase QLM immobilized either on macroporous MMA crosslinked with DVB or on cross-linked DVB polymer beads of 200-1000 microns in diameter (1 g) were mixed vigorously for 20 min Ethanol (at a ratio between ethanol and oil of 2:1 on molar basis) was added stepwise in three equivalent batches each one hour apart. The reaction medium containing the immobilized lipase preparation was shaken at 170 rpm and 30° C. Samples were withdrawn from the reaction mixture at different time intervals for determination of the enzyme activity. After 24 hours of reaction, the reaction medium was filtered off and a new fresh batch of substrates was introduced using the same batch of immobilized enzyme. Twenty consecutive reaction cycles were carried out using the same batch of immobilized lipase. FIG. 8 shows the degree of conversion of fish oil to fatty acids ethyl esters after 3 hours of reaction using the same batch of biocatalyst in 20 cycles. The results show that both different biocatalysts increased their transesterification activity by approximately 20% when the same batch of biocatalysts were used in 20 cycles. The interpretation of this result is due to removal of impurities adsorbed on the polymer resin which might mask the activity of the enzyme in early batches.

### Example 14

### Purification Process of Lipase QLM Immobilized on Different Polymer Resins for Concentration of Omega-3 Fatty Acids in the Form of Glycerides

This set of experiments was conducted to show that when Lipase QLM is immobilized on different polymer resins it might change its selectivity toward the fatty acyl groups bound to the glycerol backbone of glycerides.

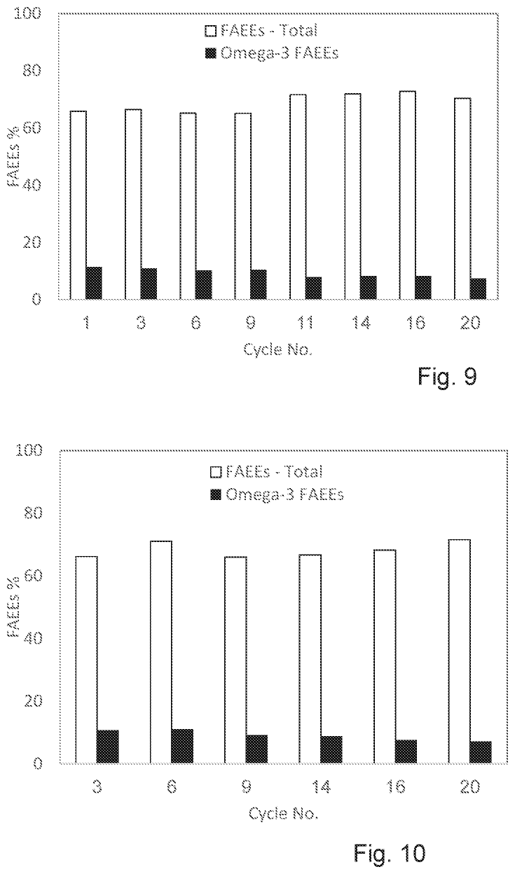

Reaction conditions: A mixture of fish oil (10 g), 3% wt. of 0.1M sodium bicarbonate solution and Lipase QLM immobilized either on macroporous MMA cross-linked with DVB or on phenol-formaldehyde cross-linked polymer beads of 200-1000 microns in diameter (1 g) were mixed vigorously for 20 min. The medium containing the immobilized lipase preparation was shaken at 170 rpm and 30° C. Ethanol (at a ratio between ethanol and oil of 2:1 on molar basis) was added stepwise in three equivalent batches each one hour apart. The reaction medium containing the immobilized lipase preparation was shaken at 170 rpm and 30° C. The conversion reaction to fatty acid ethyl esters under these conditions was allowed to proceed to 60-70% conversion, and thereafter, the reaction medium was filtered off and a new fresh batch of substrates was introduced using the same batch of immobilized enzyme. Twenty consecutive reaction cycles were carried out using the same batch of immobilized lipase. FIGS. 9 and 10 show the degree of conversion of fish oil to fatty acids ethyl esters as well as the omega-3 fatty acid ethyl after 6 hours of reaction using the same batch of the two different biocatalysts in 20 cycles. The results show that both biocatalysts, although significantly different, unexpectedly decreased their transesterification activity for omega-3 fatty acyl groups from 11.5% down to 7.4% after 6 hours of reaction, while maintaining the total conversion to fatty acids ethyl esters unchanged in the range of 65-72% when the same batches of biocatalysts were used in 20 consecutive cycles. These results indicate that when Lipase QLM is immobilized on different polymer resins, its selectivity towards omega-3 fatty acyl groups is decreased with progressing with the number of reaction cycles using the same batch of biocatalyst. Without being bound by theory, the decrease in changing of the transesterification selectivity of the biocatalyst towards the omega-3 fatty acyl groups may probably be attributed to detachment of other materials/substances interfering in the transesterification/esterification process which favor de-acylation of omega-3 fatty acyl groups off the glycerol backbone.

### Example 15

### FTIR (Fourier Transform Infra Red) Analysis for the Immobilized Lipases at Different Stages of the Treatment with the Reaction Medium Comprised of Fish Oil, Ethanol ⅔ on Molar Basis with the Fish Oil, and Sodium Bicarbonate Solution of 0.1M (3% by Weight of Oil), was Conducted

For this analysis Lipase QLM was immobilized by adsorption on a macroporous polymer resin composed of MMA crosslinked with DVB. The following FTIR spectra were obtained for the biocatalysts:
1. Before reaction (dry powdered biocatalyst).
2. After different time intervals of transesterification reaction. The biocatalyst after reaction was filtered and then powdered.
3. After different time intervals of transesterification reaction. The biocatalyst after reaction was filtered, and then washed with n-hexane to remove any adsorbed oil and other reagents and products, and thereafter dried and powdered.
4. The same batch of biocatalyst was used in several consecutive batches, each time starting with a fresh reaction medium. The biocatalyst was either filtered and powdered, or filtered, washed with n-hexane, and then dried and powdered.

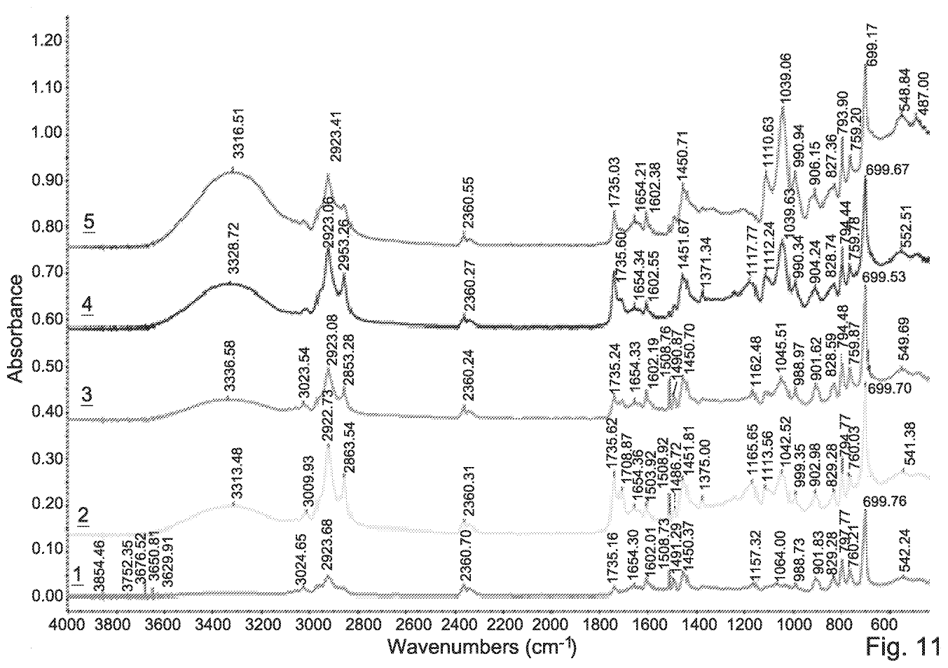

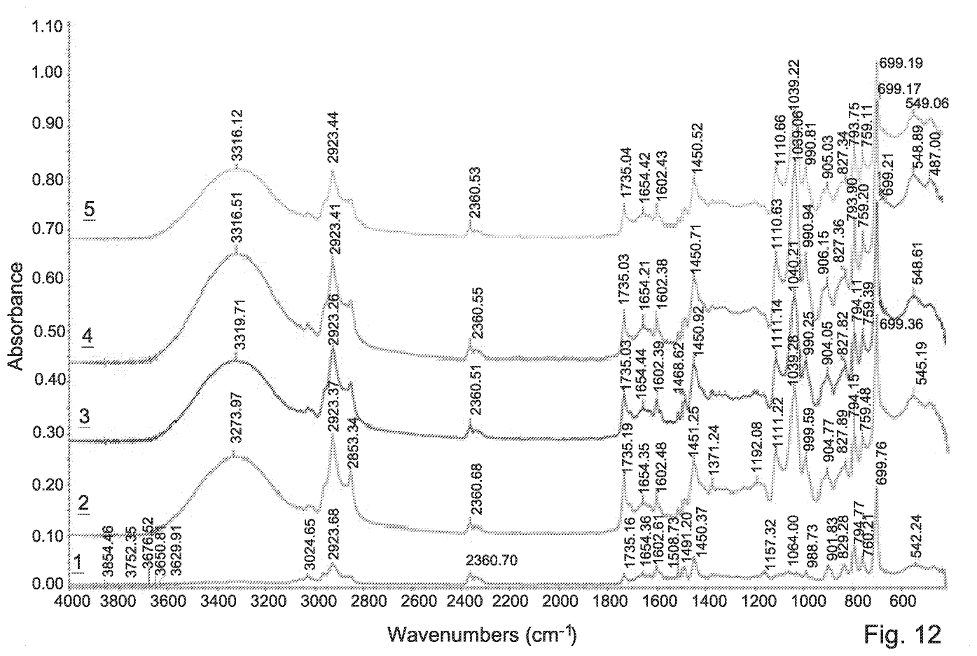

FIG. 11 shows that the FTIR spectra for Lipase QLM immobilized on the polymer resin before reaction has shown narrow bands at 2950 and 2875 cm⁻¹ corresponding to the vibrational modes of asymmetric and symmetric stretching of methyl groups, respectively. The same can be said for the bands related to stretching vibration of the carbonyl groups of the polymethylmethacrylate, to the stretching between carbon atoms in the aromatic ring at 1452 and 1512 cm⁻¹, and sharp bands at 869-682 cm⁻¹ which are characteristic of the C—H out-of-plane bending of alkyl-substituted benzene. The small bands at 1654 and 1539 cm⁻¹ which correspond to the amide I and amide II confirm the presence of small amount of lipase enzyme in all tested samples presented in FIGS. 11 and 12. The narrow bands at 1452 and 1512 cm⁻¹ confirm the presence of DVB in the structure of the biocatalysts since it can be attributed to the bond stretching between carbon atoms in the aromatic ring.

FIG. 11 shows that the FTIR spectra of Lipase QLM immobilized on the polymeric resin before reaction (1) did not show bands corresponding to N—H and O—H stretching at 3200 to 3600 cm⁻¹, however showed very modest bands corresponding to stretching vibration for the aliphatic bond C—N at 1020 to 1250 cm⁻¹ which both indicate the presence of enzyme molecules immobilized on the polymer resin. The appearance of very modest bands for the bonds C—H, C═O, Amide I and Amide II, and specifically, the C—N aliphatic amine for the dry biocatalyst sample before reaction are interpreted because of masking effects of contaminants adsorbed on the surface area of the polymer resin.

The presence of lipase can be confirmed by the bands at 1654 and 1539 cm⁻¹ that correspond to the amide I and Amide II (FIG. 11). These same bands also were observed before and after reactions in consecutive batches. This indicates that the enzyme remains adsorbed on the polymer resin when the same batch of biocatalyst is used in multiple reactions.

The bands at 3100-3600 cm⁻¹ normally corresponds either to stretching of the hydroxyl (O—H) or amine groups (N—H) where —OH can originally be in proteins, water, alcohols, free fatty acids, and amine group comes from proteins, enzymes, and alkyl amines. These bands do not appear in the FTIR spectra of the immobilized enzyme before reaction while significantly enlarged after use of enzyme in transesterification reactions.

Furthermore, it can be clearly seen in FIG. 11, that the bands at 2950 and 2875 cm⁻¹ corresponding to the vibrational modes of asymmetric and symmetric stretching of methyl groups, the bands related to stretching vibration of the carbonyl group of the polymethylmethacrylate, and to the stretching between carbon atoms in the aromatic ring at 1452 and 1512 cm⁻¹, and of major importance, the bands corresponding to stretching vibration for the aliphatic bond C—N at 1020 to 1250 cm⁻¹ which both indicate the presence of enzyme molecules, all have been significantly enlarged when progressing with the reaction time as well as with the number of cycles using the same batch of biocatalyst. These results support applicant's approach that the transesterification reaction medium including the reactants, intermediates, and the products, all contribute to desorption of undesired contaminants adsorbed on the surface area of the polymer resin during enzyme immobilization process, thereby leading to masking of the reactants from having free access to the active site of the immobilized enzyme.

It is important to observe that the ratio between the bands at 1020 and 1250 cm⁻¹ as compared to other bands can be used to verify the degree of the contamination of the immobilized enzymes with other undesired adsorbed contaminates which might be released into the reaction medium and result in contaminating and coloring the final product. The FTIR spectra for the biocatalyst before reaction, soaked in the reaction mixture for one hour with and without wash with n-hexane, as well as after several cycles of reactions using the same batch of biocatalyst without and after wash with n-hexane, suggest that a change in the enzyme/support band area ratio after the reaction, occurred in favor of the enzyme and support. Without being bound by theory, this change can be attributed to the wash-out of significant amounts of the contaminants adsorbed on the surface of the polymer resin, whereby the functional groups of the enzyme and of the support become more exposed, which increases IR absorbance.

The same phenomenon has also been observed in FIG. 12 where the above-mentioned specific bands have been significantly enlarged when the same batch of enzyme was used in multiple reaction cycles which resulted in desorption of contaminates and therefore increasing the exposure of the functional groups of the enzyme and polymer support to the source of IR radiation.

### Example 16

### Qualification and Quantification of Leachables/Extractables from the Immobilized Enzyme

In the following experiments, the leachable (extractable) materials from the polymer used for the immobilization of the enzyme were identified. Different samples of the MMA cross-linked with DVB polymer resin as well as the immobilized enzyme were subjected to different pretreatment methods and thereafter analyzed to qualify and quantify the possible components which might be leached out of the polymer resin used as a carrier for the enzyme. The procedure was applied to determine and identify the components leachable from the polymer resin used for the immobilization of the biocatalyst is described below:

A 2-gram portion of each biocatalyst sample was extracted at 78° C. for 2 hours using 20 milliliters of 95% ethanol/5% water. After the exposure period, a portion of the liquid was transferred to a 2 mL autosampler vial for analysis. Data was acquired in both scan and selective ion monitoring (SIM) mode. For the SIM mode analysis, ions of interest for each analyte were selected for use in formal quantitation. Ions of interest are listed in Table 13. Calibration was achieved using reference materials of known purity diluted in 95% ethanol/5% water. These standards were analyzed along with the sample extracts and were utilized to produce calibration curves for quantitation. All blanks, sample extracts, and calibration standards were analyzed on an Agilent 7890A gas chromatograph equipped with a 30-meter Rtx-5ms column and a 5975C mass selective detector. Table 14 shows the concentration results for the different leachable components from the polymer resin as well as from the biocatalyst once used in multiple transesterification reactions.

TABLE 13
The LOQ and LOD for the different possible ion of interest leaching from
the polymer resin used for immobilization of Lipase QLM using a GC-MS***.

| | Methyl | | Butyl | | |
|---|---|---|---|---|---|
| | methacrylate | Styrene | methacrylate | Ethylvinylbenzene | Divinylbenzene |
| SIM | 41, 69 | 78, 104 | 41, 69, 87 | 117, 132 | 115, 130 |
| Ions | | | | | |
| (m/z) | | | | | |
| LOQ* | 0.31 | 4.39 | 4.76 | 6.84 | 9.10 |
| (ppm) | | | | | |
| LOD** | 0.03 | 0.01 | 0.02 | 0.19 | 0.52 |
| (ppm) | | | | | |

LOQ*—Method limit of quantitation and,
LOD**—Limit of detection for each analyte
GCMS***—Gas Chromatography - Mass Spectrometry

TABLE 14
The concentration of different leachable from Lipase QLM immobilized
on polymethyl methacrylate cross-linked with DVB beads at different
stages of use as described below. Samples of the immobilized enzyme
were treated according to the above-mentioned procedure.

| Compound | | | | |
|---|---|---|---|---|
| extracted (ppm) | Control 1 | Control 2 | Sample 1 | Sample 2 |
| Methyl methacrylate | 70.6 | 36.7 | <10 | <10 |
| Styrene | 3.2 | 13.0 | <1 | <1 |
| n-Butyl methacrylate | 39.4 | 32.9 | <10 | <10 |
| Ethylvinylbenzene | 16.5 | 9.6 | <5 | <5 |
| Divinylbenzene | 70.6 | 38.4 | <10 | <10 |
| Total | 200.3 | 130.2 | <46 | <46 |

Control 1: Lipase QLM immobilized on methyl methacrylate cross-linked with DVB polymer resin with no treatment.
Control 2: Lipase QLM immobilized on methyl methacrylate cross-linked with DVB resin. The resin was prewashed with a mixture of alcohol (95%) and water (5%) at a ratio of 1:10, respectively.
Sample 1: Immobilized lipase after reaction cycle 1. The immobilized enzyme was first prewashed with n-hexane to remove adsorbed reaction mixture and then subjected to the above-mentioned procedure.
Sample 2: Immobilized lipase after reaction cycle 2. The immobilized enzyme was first prewashed with n-hexane to remove adsorbed reaction mixture and then subjected to the above-mentioned procedure.

The results presented in Table 14 show that when the polymer resin was used AS IS for the immobilization of the enzyme, without prewash with ethanol/water mixture the total concentration of the leachables as determined by GCMS was 200.3 ppm, while when the polymer resin was prewashed with ethanol/water mixture and then used for the immobilization of the enzyme, the total concentration of leachables was reduced to 130.2 ppm. The results in Table 14 show also that when the immobilized enzyme was used consecutively in multiple cycles of transesterification of fish oil with ethanol, the concentration of leachables was reduced significantly to less than 46 ppm in total after the first reaction cycle.

1. A method for purifying an immobilized lipase preparation comprising a reaction cycle comprising the steps of (1) reacting reaction substrates comprising at least one fatty acid source comprising oils, glycerides, free fatty acids and/or fatty acid alkyl esters (FAAE) with an alkyl alcohol, in the presence of at least 100 ppm water or aqueous alkaline buffer solution and a lipase preparation in an immobilized form, to yield fatty acid alkyl esters, free fatty acids, partial glycerides and glycerol and water as byproducts, allowing the reaction to proceed until at least part of the reaction substrate, optionally 10%, 20%, 30%, 40% or 50% w/w or more, is converted to FAAE, (2) collecting the medium by filtration to obtain a purified immobilized lipase preparation and optionally washing said purified immobilized lipase preparation with a suitable solvent, and optionally repeating said reaction cycle for 1-10 additional times, wherein each reaction cycle using a fresh reaction substrate, while using the same batch of immobilized enzyme preparation in all cycles.

2. A method for purifying an immobilized lipase preparation comprising at least one first reaction cycle, the reaction cycle comprising the steps of:
(a) providing a reaction substrate comprising at least one fatty acid source, specifically at least one of oils, fats, glycerides, free fatty acids and fatty acid alkyl esters;
(b) adding to said reaction substrate water or aqueous alkaline buffer at more than 100 ppm up to 70% w/w to form a reaction medium;
(c) providing an immobilized lipase preparation comprising a lipase immobilized on an organic or inorganic support;
(d) adding the said lipase preparation to said reaction medium to form a reaction mixture, wherein the pH of the reaction medium is a pH of 4-11 and wherein the temperature of the reaction mixture is 10-50° C.;
(e) subjecting said fatty acid source to alcoholysis by stepwise adding to said reaction mixture an alkyl alcohol, at a molar ratio of at least 2:1 between said alcohol and said reaction substrate, and stiffing or shaking or recirculating the resulting mixture until at least part of said reaction substate, optionally at least 10%, 20%, 30%, 40% or 50% of said reaction substrate are converted to fatty acid alkyl esters;
(f) adding alkyl alcohol to the reaction mixture of step (e) to reach at least a total molar ratio of 3:1 between said alcohol and said reaction substrate where said support is a hydrophobic or mild hydrophobic polymer resin, and less than 2:1 where said support is a hydrophilic polymer resins used as carrier for the immobilization of the enzyme, and allowing the reaction to proceed until conversion of said reaction substrate to fatty acid alkyl esters exceeds 70%;
(g) collecting the reaction medium of step (f) by filtration and keeping the immobilized lipase preparation, and optionally washing the immobilized lipase preparation with a suitable solvent,
wherein the collected reaction medium filtrate separates into two phases, an upper (lighter) oil phase comprising formed fatty acids alkyl esters preferably at more than 50% w/w, free fatty acids and mono-, di- and residual tri-glycerides preferably at less than 10% w/w, and a lower (heavier) phase comprising the formed glycerol and water, wherein the upper oil phase comprises extracted hydrophobic pigment components and other hydrophobic contaminants comprised in the said immobilized lipase preparation, such as silicon compounds and/or desorbed components of the support, the lower phase comprises extracted hydrophilic pigment components and other hydrophilic contaminants comprised in the said immobilized lipase preparation, and any reaction intermediate products including free fatty acids, fatty acids soaps, mono- and di-glycerides comprise extracted amphiphilic pigment components and other amphiphilic contaminant comprised in the said immobilized lipase preparation; and measuring the color intensity in said upper oil phase and in said lower phase by suitable means;
(h) repeating said reaction cycle comprising said steps (a)-(g) at least once in a subsequent identical reaction cycle, preferably repeating said reaction cycle 1-10 times, using in each repeated reaction cycle the same batch of said lipase preparation provided in step (c) of the first cycle, with fresh reaction substrate and alkyl alcohol in each cycle, until the color intensity of the oil phase of said filtrate is reduced to a desired level as compared to the color intensity of the oil phase obtained in said first reaction cycle, wherein the immobilized lipase collected after the at least one subsequent reaction cycle is of higher purity and of at least comparable enzymatic activity to that of the immobilized lipase preparation provided in step (c) of said first cycle.

3. The method of claim 1 or 2, wherein said fatty acid source is at least one oil, such as a plant oil such as soybean oil, canola oil, rapeseed oil, olive oil, MCT oil, castor oil, palm oil, sunflower oil, safflower oil, peanut oil, cotton seed oil, Jatropha oil, coconut oil or corn oil; algal oil, fish oil, oleaginous microorganisms derived oil; waste cooking oil; and any mixtures thereof; said fat is animal-derived fat or brown grease; said free fatty acids are saturated or unsaturated fatty acids of 12-20 carbon atoms, such as mono- or polyunsaturated fatty acids and short and medium-chain fatty acids of 2-12 carbon atoms; said glycerides are mono-, di- and triglycerides of short-, medium- and long-chain fatty acids of 12-20 carbon atoms, and their mixtures at any ratio; and said fatty acid alkyl esters are at least one of methyl, ethyl or longer alkyl fatty acid esters, wax esters and sterol esters.

4. The method of any one of claims 1 to 3, wherein said alkyl alcohol is short-chain C₁₋₆ alkyl alcohol, preferably ethanol, medium-chain C₈₋₁₂ alkyl alcohol, or long-chain C₁₄₋₂₂ alkyl alcohol.

5. The method of any one of claims 1 to 4, wherein said lipase is any one of lipase derived from Rhizomucor miehei, Pseudomonas sp., Pseudomonas cepacia, Pseudomonas fluorescens, Rhizopus niveus, Mucor javanicus, Rhizopus oryzae, Aspergillus niger, Penicillium camembertii, Burkholderia ubonensis (strain PL266-QLM, also referred to herein as Lipase QLM) and cepacia, Alcaligenes sp., Acromobacter sp., Burkholderia sp., Thermomyces lanuginosa, Humicola lanuginosus, Chromobacterium viscosum, Candida antarctica B, Hyphozyma sp., Candida parapsilosis, Candida rugosa, Candida antarctica A, H. insolens, Crytococcus spp., Geotricum candidum Pseudomonas (Burkholderia) cepacia, Pseudomonas stutzeri or papaya seeds, and pancreatin.

6. The method of any one of claims 1 to 4, wherein said lipase is a random or sn-1,3 positional specific lipase.

7. The method of any one of claims 1 to 6, wherein said lipase possesses selectivity toward a specific type of fatty acids such as short-, medium- and long-chain fatty acids, or saturated-, mono-unsaturated or polyunsaturated fatty acids.

8. The method of any one of claims 1 to 7, wherein said support is an anion- or cation-exchange resin, a hydrophilic organic polymer, such as polyacrylate, polymethyl methacrylate, polymethyl methacrylate or cross-linked phenol formaldehyde condensate, or hydrophobic/mild hydrophobic organic polymer such as polyvinyl alcohol, polydivinyl-benzene and polystyrene, and any mixture thereof, an inorganic support such as silica, Celite, diatomaceous earth and perlite.

9. The method of claim 8, wherein said support is an anion- or cation-exchange resin, a hydrophilic organic polymer, such as polyacrylate, polymethyl methacrylate, polymethyl methacrylate or cross-linked phenol formaldehyde condensate, or hydrophobic organic polymer such as polyvinyl alcohol, polydivinylbenzene and polystyrene, and any mixture thereof and said desorbed component of the support is monomer/s or oligomer/s of said resin/polymer.

10. The method of any one of claims 1 to 9, wherein said immobilized lipase preparation is in powder form with particle size of 1-100 microns, or in the form of beads, typically of 0.01-2 mm diameter.

11. The method of any one of claims 1 to 10, wherein said aqueous alkaline buffer is a bicarbonate, carbonate, acetate, phosphate, citrate or tris, buffer salt or any of their combination.

12. The method of any one of claims 1 to 11, wherein said water or aqueous alkaline buffer is added to said reaction substrate at an amount of from about 1%, 2%, 3%, 4%, 5%, 10%, or 20% of weight of said fatty acid source.

13. The method of any one of claims 1-12, wherein said lipase is added to said reaction medium at 0.1-20% wt. based on weight of said fatty acid source, such as 1, 2, 3, 4, 5, 6, 7, 10 and up to 20% wt.

14. The method of any one of claims 1 to 13, wherein said fatty acid source comprises oils and the products of the enzymatic transesterification reaction are fatty acid alkyl esters and glycerol as byproduct.

15. The method of any one of claims 1 to 13, wherein said fatty acid source comprises free fatty acids, and the products of the enzymatic esterification reaction are fatty acid alkyl esters and water as byproduct.

16. The method of any one of claims 1 to 13, wherein said fatty acid source comprises fatty acids alkyl esters, and the alcoholysis reaction products are different fatty acids alkyl esters and an alcohol as a byproduct.

17. The method of any one of claims 1 to 13, wherein said fatty acid source comprises a mixture of fatty acids and triglycerides, at any ratio, and the esterification/transesterification reaction products are fatty acids alkyl esters, and glycerol and water as byproducts.

18. The method of any one of claims 1 to 18, wherein said fatty acid source is a mixture of fatty acids and mono-, di-, and tri-glycerides at any ratio, and the esterification/transesterification reaction products are fatty acids alkyl esters, and glycerol and water as byproducts.

19. The method of any one of claims 1 to 13, wherein said fatty acid source is an isolated mono-, di-, or tri-glyceride, or any mixture of at least two thereof, and the transesterification reaction products are fatty acids alkyl esters, and glycerol as byproduct.

20. The method of any one of claims 1 to 13, wherein said fatty acid source comprises a mixture of triglycerides, free fatty acids and lecithin gum, at any ratio, and the esterification/transesterification reaction products are fatty acids alkyl esters, and glycerol water, lyso-phospholipids and glycerophospholipids as byproducts.

21. The method of any one of claims 1 to 17, wherein said color intensity of said upper oil phase, optionally after dilution with an organic solvent, is measured by optical density (OD) at 420 nm

22. The method of claim 21, wherein said dilution of oil phase:solvent is at a ratio of 0-10 v/v, depending on the optical density.

23. The method of claim 21 or claim 22 wherein said organic solvent is any one of n-hexane, iso-propanol, n-propanol, n-butanol, iso-butanol and tert-butanol.

24. The method of any one of the preceding claims, wherein said subsequent reaction cycle is repeated from 2 to 3 times, 2 to 4 times, 2 to 5 times, 2 to 6 times and up to about 10 times.

25. A purified immobilized lipase preparation obtained by the method of any one of claims 1 to 24, in which the lipase is immobilized on a macroporous resin polymer that is a hydrophobic polymer, a mild hydrophobic polymer or a mixed hydrophobic/hydrophilic polymer.

26. A purified immobilized lipase preparation, in which the lipase is immobilized on a macroporous resin polymer that is a hydrophobic polymer, a mild hydrophobic polymer or a mixed hydrophobic/hydrophilic polymer, wherein the optical density of the product of a reaction between a fatty acid source and an alcohol in a reaction medium containing water or alkaline buffer in the presence of said purified lipase is reduced compared to optical density of the product of the same reaction carried out in the presence of an identical immobilized lipase preparation that is not purified.

27. A purified immobilized Lipase QLM preparation in which the lipase is immobilized on a macroporous resin polymer that is a hydrophobic polymer, a mild hydrophobic polymer or a mixed hydrophobic/hydrophilic polymer, wherein the optical density at 420 nm of the oil phase of the product of soybean oil treated by one reaction 24 hours cycle with ethanol at an oil to ethanol molar ratio of 1:3 in the presence of said immobilized lipase at a concentration of 10% w/w is 0.150 OD, and 0.100 OD after three reaction cycles each of 24 hours using the same batch of said immobilized lipase, compared to optical density of 0.750 OD and 0.360 OD for soybean oil mixed with 10% w/w immobilized lipase QLM preparation for a first cycle of 24 hours and for three cycles of 24 hours each with same batch of lipase.

28. The purified immobilized Lipase QLM preparation of claim 27, wherein the optical density at 420 nm of the oil phase of the product of MCT oil treated by one reaction cycle of 24 hours with ethanol at an oil to ethanol molar ratio of 1:3, in the presence of said immobilized lipase at a concentration of 10% w/w is 0.150 OD and 0.050 OD after three reaction cycles each 24 hours when using the same batch of immobilized enzyme, compared to 0.530 OD and 0.082 OD for MCT oil mixed with 10% w/w immobilized lipase QLM for a first cycle of 24 hours and for three cycles each of 24 hours and each with the same batch of lipase.

29. The purified immobilized Lipase QLM preparation of claim 27, wherein the optical density at 420 nm of the oil phase of the product of fish oil treated by one reaction cycle of 24 hours with ethanol at an oil to ethanol molar ratio of 1:3 in the presence of said immobilized lipase at a concentration of 10% w/w is 0.7 OD and 0.32 OD after three reaction cycles each 24 hours when using the same batch of immobilized enzyme, compared to 0.86 OD and 0.50 OD for fish oil mixed with 10% w/w immobilized lipase for a first 24 hours cycles and for three 24 hours cycles each with the same batch of lipase.

30. A purified immobilized Lipase QLM preparation in which the lipase is immobilized on a macroporous resin polymer composed of polymethyl methacrylate crosslinked with divinyl benzene (DVB) having the FTIR spectra designated (2), (3), (4) or (5) in FIG. 11 and/or in FIG. 12.

31. A purified immobilized lipase preparation of claim 26, wherein said lipase is any one of Rhizomucor miehei, Pseudomonas sp., Pseudomonas cepacia, Pseudomonas fluorescens, Burkholderia ubonensis (strain PL266-QLM) and cepacia, Alcaligenes sp., Burkholderia sp., Thermomyces lanuginosa, Humicola lanuginosus, Candida antarctica B, Hyphozyma sp., Candida parapsilosis, Candida antarctica A, Pseudomonas (Burkholderia) cepacia and Pseudomonas stutzeri, immobilized on a hydrophobic linear or branched aromatic or hydrophobic aliphatic polymer-based support.

32. A purified lipase preparation of any one of claims 24 to 31, wherein said mild hydrophobic polymer is divinylbenzene.

33. A purified lipase preparation of any one of claims 24 to 31, wherein said mixed hydrophobic/hydrophilic polymer is cross linked divinylbenzene-methylmethacrylate polymer.

34. A purified immobilized lipase preparation of any one of claims 24 to 33, for use as a biocatalyst in enzymatic interesterification, transesterification, esterification, alcoholysis, amidation, transamidation and/or hydrolysis reaction, wherein the final products of said reactions are essentially free of coloration and/or contamination such as silicon compounds and/or any monomer or oligomers desorbed from the enzyme polymer support.

35. A purified lipase preparation of any one of claims 24 to 34, wherein said purified lipase exhibits equivalent or higher catalytic activity in enzymatic processing and re-forming of oils and fats, including of interesterification of oils and fats and transesterification of oil glycerides and an alkyl alcohol for production of partial glycerides, fatty acids alkyl esters and glycerol at any predetermined ratio.

36. A process of enzymatically producing omega-3 fatty acids concentrates comprising enzymatic transesterification omega-3 containing oils, that may be derived from fish, plant and/or oleaginous microorganisms with a short-chain alkyl alcohol in the presence of a purified immobilized lipase as defined in any one of claims 24 to 33, wherein said concentrates have low color intensity and low levels of other contaminants such as silicon compounds and/or any monomer or oligomer desorbed from the enzyme polymer support.

37. A process of producing re-formed interesterified oils and fats having low color intensity and low content of contaminants such as silicon compounds and any monomer or oligomer desorbed from the enzyme polymer support, by any one of enzymatic interesterification, transesterification, esterification, alcoholysis, amidation, transamidation and/or hydrolysis reaction, using a purified immobilized lipase preparation as defined in any one of claims 24 to 33.
